(19) United States
(12) Patent Application Publication
(10) Pub. No.: US 2013/0189253 A1
(43) Pub. Date: Jul. 25, 2013

(54) MODULATION OF SIRP-ALPHA - CD47 INTERACTION FOR INCREASING HUMAN HEMATOPOIETIC STEM CELL ENGRAFTMENT AND COMPOUNDS THEREFOR

(71) Applicants: University Health Network, Toronto (CA); The Hospital For Sick Children, Toronto (CA)

(72) Inventors: Jayne Danska, Toronto (CA); John E. Dick, Toronto (CA); Tatiana Prasolava, Toronto (CA); Katsuto Takenaka, Fukuoka (JP)

(73) Assignees: THE HOSPITAL FOR SICK CHILDREN, Toronto, ON (CA); UNIVERSITY HEALTH NETWORK, Toronto, ON (CA)

(21) Appl. No.: 13/663,801

(22) Filed: Oct. 30, 2012

Related U.S. Application Data
(63) Continuation of application No. 12/682,307, filed on May 21, 2010, now abandoned.
(60) Provisional application No. 60/960,724, filed on Oct. 11, 2007.

(51) Int. Cl. A61K 39/395; A61K 38/17

(52) U.S. Cl. CPC A61K 39/3955; A61K 38/1709
USPC 424/134.1; 514/21.2; 424/172.1; 530/387.3

(57) ABSTRACT

The invention relates to modulating the SIRPα-CD47 interaction in order to increase hematopoietic stem cell engraftment and compounds therefor. In some embodiments, there is provided isolated SIRPα and CD47 polypeptides, fragments and fusion proteins for enhancing hematopoietic stem cell engraftment. Further there is provided methods for increasing hematopoietic stem cell engraftment through administration of the above polypeptides.

### CROSS-REFERENCE TO RELATED APPLICATIONS

The present application is a continuation application of a co-pending U.S. application Ser. No. 12/682,307, filed May 21, 2010, the contents of which are incorporated herein by reference in its entirety, which is a 35 U.S.C. §371 National Phase Entry Application of International Application PCT/CA2008/001814 filed Oct. 10, 2008, which designated the U.S., and claims the benefit under 35 U.S.C 119(e) of U.S. Provisional Patent Application Ser. No. 60/960,724 filed on Oct. 11, 2007.

### SEQUENCE LISTING

The instant application contains a Sequence Listing which has been submitted in ASCII format via EFS-Web and is hereby incorporated by reference in its entirety. Said ASCII copy, created on Apr. 8, 2010, is named Sequence_Listing_txt.txt and is 49,152 bytes in size.

### FIELD OF THE INVENTION

The invention relates to modulating the SIRPα-CD47 interaction in order to increase hematopoietic stem cell engraftment and compounds therefor. In some embodiments, there is provided isolated SIRPα and CD47 polypeptides, fragments and fusion proteins for enhancing hematopoietic stem cell engraftment. Further there is provided methods for increasing hematopoietic stem cell engraftment through administration of the above polypeptides.

### BACKGROUND OF THE INVENTION

The transplantation of human hematopoietic stem cells (HSC) from bone marrow (BM) or G-CSF mobilized peripheral blood (PB) has been one of the most important clinical applications of stem cell biology. HSC transplantation in individuals with neoplastic disease enables the use of a high dose chemotherapy regimen and subsequent HSC rescue to overcome the resultant hematopoietic failure due to chemotherapy, enhancing cure rates for both hematologic and non-hematologic tumors. Additionally, genetic diseases such as thalassemia and certain immune-deficiencies can be managed by autologous transplantation of gene-corrected HSC or by transplantation of allogeneic HSC. A key discovery enabling successful HSC transplantation was identification of the human leukocyte antigen (HLA) system as the human major histocompatibility complex. Although HLA disparity between donor and host plays a major role in graft rejection, graft failure can occur even in patients receiving an unmanipulated, HLA-identical transplant (Thomas, E. D. et al. (1977) Blood 49, 511-33; Storb, R. et al. (1977) N Engl J Med 296, 61-6). Graft rejection in this setting may be related in part to mismatch at minor histocompatibility antigens (Gale, R. P. et al. (1981) Blood 57, 9-12). Additional genes other than the HLA haplotypes that modulate HSC engraftment have not been characterized.

HSC reside in supportive microenvironmental niches comprised of fibroblastic stroma, osteoblasts, osteoclasts, macrophages, and endothelial cells (Suda, T. et al. (2005) Trends Immunol 26, 426-33). Abrogation of HSC interaction with such niches, for example by blocking specific adhesion proteins or chemokine receptors, prevents HSC engraftment (Lapidot, T. et al. (2005) Blood 106, 1901-10). Additionally, natural killer (NK) cells and macrophages, both components of the innate immune system, have been shown to play a role in murine HSC transplantation (Murphy, W. J. et al. (1987) J Exp Med 165, 1212-7) and human hematopoietic xenotransplantation (McKenzie, J. L. et al. (2005) Blood 106, 1259-61), respectively. A better understanding of the mechanisms underlying hematopoietic stem cell engraftment following transplantation, including genes that control regulatory pathways, could ultimately translate into better clinical outcomes.

Xenotransplantation in the non-obese diabetic/severe combine immune-deficient NOD.Prdck^{sc/sc} (NOD.SCID) mouse has become the “gold standard” assay for human HSC engraftment. The assay is based on the ability of human HSC to repopulate the immune system of these animals following intravenous injection (Wang, J. C. Y. et al. “Normal and leukemic human stem cells assayed in immune-deficient mice” in: Hematopoiesis—A Developmental Approach (ed. Zon, L. I.) 99-118 (Oxford University Press, New York, USA, 2001). The cells that initiate the human xenograft are operationally defined as SCID-repopulating cells (SRC), possess properties attributed to HSC, and are distinct from more mature progenitors assayed in vitro. This system has enabled analysis of the proliferation, differentiation, and self renewal properties of SRC within the human HSC compartment.

Signal regulatory proteins (SIRPs) constitute a family of cell surface glycoproteins which are expressed on myeloid (including macrophages, granulocytes, myeloid dendritic cells, and mast cells) and neuronal cells (summarized in Barclay, A. N. & Brown, M. H., Nat Rev Immunol 6, 457-64 (2006); see also WO 97/48723). SIRPs constitute a diverse multigene family of immune receptors encompassing inhibitory (SIRPα), activating (SIRPβ), nonsignaling (SIRPγ) and soluble (SIRPδ) members. CD47, a broadly expressed transmembrane glycoprotein, functions as a cellular ligand for SIRPα and binds to the NH₂-terminal extracellular terminus of SIRPα. SIRPα's role has been best documented in respect of its inhibitory role in the phagocytosis of host cells by macrophages. In particular, the binding of SIRPα on macrophages by CD47 expressed on target cells, generates an inhibitory signal that negatively regulates phagocytosis. However, more recent findings have also demonstrated additional positive signaling effects mediated through SIRPα binding (Shultz, L. D. et al. (1995) J Immunol 154, 180-91).

A variety of approaches have been proposed to disrupt the CD47-SIRPα interaction in an effort to effect a biological outcome. These encompass the use of fragmented/truncated SIRPα and/or CD47 proteins and antibodies thereto (see, for example, International Patent Application Publication No. WO 99/40940; U.S. Patent Application Publication Nos. 2003/0026803 and 2006/0135749; and U.S. Pat. No. 6,913,894), for modifying immune function (see, for example, International Patent Application Publication No. WO 99/40940; and U.S. Patent Application Publication Nos. 2003/0026803 and 2007/0113297), and for xenotransplantation (see, for example, U.S. Patent Application Publication Nos. 2005/0255550, and 2007/0157328).

There remains a need to identify molecules capable of supporting HSC engraftment and hematopoiesis upon transplantation.

### SUMMARY OF THE INVENTION

According to one aspect, there is provided an isolated polypeptide selected from the group consisting of a polypeptide consisting of a) the amino acid sequence of SEQ ID NO. 1; b) a polypeptide consisting of a fragment of the amino acid sequence of SEQ ID NO. 1, wherein the fragment comprises at least one of residues 31, 32, 34, 37, 74, 77, 83, 84, 86, 87, 90, 91, 96, 100, 102, 114, 118, 126 of SEQ ID NO. 1; and c) one of the polypeptide in a) and b) with up to 1 amino acid insertion, deletion or substitution for every 7 amino acids in length of the polypeptide, wherein the polypeptide comprises at least one of residues 31, 32, 34, 37, 74, 77, 83, 84, 86, 87, 90, 91, 96, 100, 102, 114, 118, 126 of SEQ ID NO. 1 and binds human CD47.

According to a further aspect, there is provided an isolated polypeptide selected from the group consisting of a) a polypeptide consisting of the amino acid sequence of SEQ ID NO. 2; b) a polypeptide consisting of a fragment of the amino acid sequence of SEQ ID NO. 2; and c) one of the polypeptide in a) and b) with up to 1 amino acid insertion, deletion or substitution for every 7 amino acids in length of the polypeptide; wherein i) at least one of residues at positions 31, 32, 34, 37, 74, 77, 83, 84, 86, 87, 90, 91, 96, 100, 102, 114, 118, 126 of SEQ ID NO. 2 in the polypeptide is replaced with corresponding residues 31, 32, 34, 37, 74, 77, 83, 84, 86, 87, 90, 91, 96, 100, 102, 114, 118, 126 of SEQ ID NO. 1; or ii) at least one of residues 129 and 130 of SEQ ID NO. 2 in the polypeptide is deleted.

According to a further aspect, there is provided an isolated polypeptide selected from the group consisting of a) a polypeptide consisting of an amino acid sequence selected from the group consisting of SEQ ID NOS. 4-13; b) a polypeptide consisting of a fragment of an amino acid sequence selected from the group consisting of SEQ ID NOS. 4-13; and c) one of the polypeptide in a) and b) with up to 1 amino acid insertion, deletion or substitution for every 7 amino acids in length of the polypeptide, wherein the polypeptide binds human CD47.

According to a further aspect there is provided a polypeptide capable of binding to human Sirpα; and antibodies to human Sirpα. Preferably, the polypeptide is the extracellular domain of human CD47 fused to the Fc portion of IgG. According to a further aspect, there is provided a pharmaceutical composition comprising a polypeptide described herein and a pharmaceutically acceptable carrier.

According to a further aspect, there is provided a method for increasing hematopoietic stem cell engraftment in a mammal comprising administering to the mammal a therapeutically effective amount of a polypeptide described herein. According to a further aspect, there is provided a use of a polypeptide described herein for increasing hematopoietic stem cell engraftment in a mammal or in the preparation of a medicament for increasing hematopoietic stem cell engraftment in a mammal.

According to a further aspect, there is provided a polypeptide described herein for increasing hematopoietic stem cell engraftment in a mammal.

According to a further aspect, there is provided an isolated nucleic acid comprising a sequence that encodes a polypeptide described herein.

According to a further aspect, there is provided an expression vector comprising a nucleic acid described herein.

According to a further aspect, there is provided a cultured cell comprising a vector described herein.

According to a further aspect, there is provided a method of producing a polypeptide comprising culturing a cell described herein under conditions permitting expression of the polypeptide.

According to a further aspect, there is provided a method for increasing hematopoietic stem cell engraftment in a human comprising modulating the interaction between human Sirpα and human CD47.

According to a further aspect, there is provided a method of identifying a compound that increases hematopoietic stem cell engraftment in a human comprising a) contacting at least one of the extracellular domain of human Sirpα and human CD47 with at least one test compound; b) determining the at least one test compound as binding to the at least one of human Sirpα and human CD47; c) contacting the test compound with human hematopoietic cells in a stromal environment; and d) determining whether hematopoietic stem cell engraftment is increased in the presence of the test compound.

According to a further aspect, there is provided a method of determining genetic polymorphisms in humans affecting hematopoietic stem cell engraftment comprising a) sequencing the Sirpα gene from a plurality of humans having undergone hematopoietic transplantation; b) determining nucleotide differences in the Sirpα gene within the plurality of humans; and c) correlating the nucleotide differences with hematopoietic stem cell engraftment to determine relevant polymorphisms.

According to a further aspect, there is provided a method of determining likelihood of hematopoietic stem cell engraftment in a recipient comprising a) sequencing the Sirpα gene from the recipient; and b) determining whether the relevant polymorphisms exist.

### BRIEF DESCRIPTION OF FIGURES

These and other features of the preferred embodiments of the invention will become more apparent in the following detailed description in which reference is made to the appended drawings wherein:

FIGS. 1A-1C illustrate the support of human hematopoiesis in vivo and in vitro in the NOD strain background. (1A) Southern blot analysis of DNA extracted from the BM of mice four weeks post-transplant with human BM (top panel; cell dose: RAG-2 knockout strains 40×10⁶ mononuclear cells, SCID and NOD/SCID mice 20×10⁶ cells) or CB (bottom panel, 15×10⁶ mononuclear cells per recipient) cells. The level of human cell engraftment was determined by comparing the intensity of the 2.7 kb α-satellite band (arrow) in the sample lanes with the control human/mouse DNA mixtures. (1B, 1C) Number of CFC generated in chimeric LTC of human Lin⁻ CB cells on irradiated mouse BM stroma. BM cells from NOD, NOR, B6, ICR, C3H, and BALB/c male mice as indicated were cultured for 5 weeks followed by irradiation and subsequent inoculation with 1.1×10⁵ (b) or 0.6-6.0×10⁵ (c) Lin⁻ CB cells. After 5 weeks of culture, cells were harvested and the total number of CFC was determined in standard clonogenic methylcellulose assays. The data is shown as the mean of 3 to 6 independent experiments.

FIGS. 2A-2B illustrate the effects of strain-specific differences at the Idd13 locus on support of human hematopoietic engraftment in vitro and in vivo. (2A) Generation of CFC in chimeric LTC of human Lin⁻ CB cells on mouse stroma from NOD congenic (NOD.NOR-Idd4, NOD.NOR-Idd5, NOD.NOR-Idd9, NOD.NOR-Idd13), NOR congenic (NOR.NOD-Idd13), and B6.NOD-Idd13 mice. Murine BM stromal layers were established as described in FIG. 1b and seeded with 0.36-2.0×10⁵ human Lin⁻ CB cells. After 5 weeks of culture, cells were harvested and the number of CFC was determined by plating in methylcellulose assays. In each experiment, NOD stroma was used as a positive control, and B6 and NOR stromal cells were used as negative controls. The data is normalized to the number of CFC generated in NOD cultures (=100%), and is shown as the mean of at least 3 independent cultures. BB, Idd13 homozygous B6 BM stroma; NN, Idd13 genotype homozygous NOD BM stroma. (2B) Eight- to 9-week-old NOD.SCID (n=11) or NOD.NOR-Idd13.SCID (n=8) mice were irradiated with 350 cGy and injected intravenously with 0.37-1.5×10⁵ Lin⁻CD34⁺ cells). Six to 7 weeks post-transplantation, mice were sacrificed and the recipients' BM was assessed for human CD45⁺ cell engraftment by flow cytometry.

FIGS. 3A-3B illustrate the identification of the critical region of Idd13 associated with support of human hematopoiesis. (3A) Graphical representation of chromosome 2 in various congenic strains on both the NOD and NOR backgrounds. Genetic mapping of the candidate interval is denoted by the striped bars to the right of the chromosomal representations. Formal congenic strain designations are as follows: 1. NOD.NOR-D2Jyh443-D2Mit452, 2. NOR.NOD-D2Jyh443-D2Jyh1493, 3. NOR.BN-D2Jyh443-D2Jyh 1493, 4. NOR.NOD-D1Ngul46-Ramp1/D2Jyh443-D2Jyh1192, 5. NOD.NOR-D2Jyh443-D2Gul482, 6. NOD.NOR-D2Jyh443-D2Jyh3941, 7. NOR.NOD-D1Ngul46-Ramp1/D2Gul188-D2Jyh1493, 8. NOR.BN-D2Jyh767-Fliz1. (3B) Generation of CFC in chimeric LTC of human Lin⁻ CB cells on mouse stroma from NOD or NOR Idd13 subcongenic mice depicted in a. Cultures performed and data expressed as in FIG. 2a. Strain designations as described in a.

FIG. 4 illustrates protein sequence alignment of SIRPα. cDNA prepared from BM-derived macrophages was used as a template for PCR amplification of Sirpα transcripts from NOD and NOR mice. The B6 mouse sequence is from the EnsEMBL database. Protein domains are indicated by open boxes.

FIGS. 5A-5B illustrate immunoblot analysis of SIRPα in BM-derived macrophages from NOD, NOR, and congenic NOR.NOD-Idd13 mouse strains. (5A) Lysates prepared from BM-derived macrophages, either untreated or treated with LPS (100 ng/ml) and IFN-γ (10 ng/ml), were subjected to immunoblot analysis using polyclonal antibodies directed against the cytoplasmic domain of SIRPα, or glyceraldehyde-3-phosphate dehydrogenase (GAPDH) as a loading control. (5B) Lysates prepared from BM-derived macrophages were incubated in the absence (−) or presence (+) of N-glycosidase F for 12 hours at 37° C. and then subjected to immunoblot analysis using polyclonal antibodies directed against the cytoplasmic domain of SIRPα. N-glycosidase F treated and untreated samples were co-electrophoresed. Molecular size standards are indicated in kilo Daltons (kD).

FIGS. 6A-6C illustrate Sirpα modulation of murine macrophage-mediated suppression of human hematopoiesis. (6A) Macrophage-mediated suppression of human hematopoiesis in LTC. MS-5 cells were seeded into 96-well tissue culture plates (2000 cells/well). Peritoneal macrophages harvested from NOD, NOR, and NOR.NOD-Idd13 heterozygous mice were seeded at doses of 200-20,000 cells/well (5 replicates per dose) with addition of 2000 human Lin⁻ CB cells per well the next day. After 3-4 weeks of culture, cells were harvested and the number of human CFC was determined by plating in methylcellulose assays. Error bars represent standard deviations. (6B) Western blot analysis showing SIRPα expression in Jurkat cells and NOR BM-derived macrophages before and after infection with control virus (CEP) or virus expressing NOD-derived Sirpα (SIRP). Protein lysates were immunoblotted with polyclonal antibody directed against SIRPα. Lane 1: Jurkat cells; lane 2: Jurkat cells infected with CEP; lane 3: Jurkat cells infected with SIRP; lane 4: NOR macrophages; lane 5: NOR macrophages infected with SIRP; lane 6: NOD macrophages. Infection with SIRP lentivirus resulted in high level expression of NOD SIRPα in NOR macrophages (lane 5). (6C) Effect of Sirpα on macrophage-mediated suppression of human hematopoiesis. NOR BM-derived macrophages were infected with control (NOR-CEP) lentivirus or lentivirus expressing the NOD-derived Sirpα gene (NOR-NOD SIRP) prior to seeding onto established MS-5 stromal cultures at doses of 20 to 20,000 cells/well (5 replicates per dose). 2000 Lin⁻ CB cells/well were added the next day and human CFC generated after 3.5 weeks in culture were assayed as described. Support of human hematopoiesis in NOR-NOD SIRP wells was significantly better than in NOR-CEP wells (P=0.032 at 20 cells/well; P=0.008 at 2000 and 20,000 cells/well). Error bars represent standard deviations.

FIGS. 7A-7E illustrate conferral of enhanced cross species reactivity with human CD47 by NOD SIRPα. (7A-7D) Flow cytometric analysis of human CD47 (hCD47) binding to murine SIRPα on BM-derived macrophages from NOD and NOD.NOR-Idd13 congenic mice. (7A) Histogram of CD11b expression; horizontal bar indicates CD11b⁺ gate. Plots in b through d are gated on CD11b. (7B) Two dimensional contour plots showing SIRPα and hCD47-Fc staining. (7C) Histogram of SIRPα expression (grey) compared to isotype control (black). (7D) Histogram of hCD47-Fc staining (grey) overlaid with human IgG-Fc control (black). (7E) Immunoprecipitation of murine SIRPα with hCD47-Fc. Protein extracts from BM-derived macrophages from NOD, NOR and NOD.NOR-Idd13 congenic mice were electrophoresed on SDS-PAGE and immunoblotted with polyclonal anti-SIRPα antibody. Each panel displays total lysate, immunoprecipitates with control human IgG-Fc protein (IP hFc) and immunoprecipitates with hCD47-Fc fusion protein (IP hCD47-Fc). The top panel compares NOD and NOR extracts, and the bottom panel compares NOD and NOD.NOR-Idd13 (Idd13cg) extracts.

FIGS. 8A and 8B illustrate protein sequence alignments of murine and human SIRPα IgV domains. (8A) cDNA prepared from BM macrophages was used as a template for PCR amplification of Sirpα transcripts from NOD and NOR mice. The C57BL/6 (B6), BALB/c and 129/Sv sequences were obtained from EnsEMBL and NCBI databases. Open boxes represent b-pleated sheets identified in the X-ray crystal structure of SIRPα and correspond to similar regions in the Ig heavy chain variable region. Amino acids that vary between mouse strains are shaded. B6 was used as the parental sequence. (8B) Exon 3 of SIRPα containing the IgV domain was PCR amplified from genomic DNA of 37 individuals from the human HapMal phase 1 release. Open boxed regions and shaded amino acids represent the same features as in (a), with V1 serving as the parental sequence. The residue marked * in an orthologous position between species, is polymorphic between NOD and other strains, and between humans. The residues marked + are polymorphic in humans and reside on the “top” solvent exposed surface of the protein where CD47 is predicted to bind.

FIG. 9 illustrates the validation of the discrimination power of three SNP assays by independent sequencing of the SIRPα IgV domain PCR amplified from each of the human HapMap samples shown (left side of the table).

FIGS. 10A and 10B illustrate the CD47 protein including extracellular IgV-like loop, five membrane spanning regions and short cytoplasmic tail (10A) CD47 (10B) CD47-IgGFc fusion.

FIGS. 11A and 11B illustrate (11A) a histogram depicting protein production in the supernatant of cultured cells transfected with mCD47-Fc and two hCD47-Fc constructs prepared with different plasmid backbones (pcDNA and pIAP369) and (11B) an immunoblot of an SDS-PAGE depicting the fusion proteins following purification from culture supernatant.

FIGS. 12A and 12B illustrate (12A) the introduction of a Kozak sequence into the pIAP369 plasmid construct containing mCD47-Fc and (12B) the sequence hCD47-Fc inserted into the pIAP369 plasmid, showing the Kozak consensus, hCD47 fragment, linker and Fc.

FIGS. 13A and 13B illustrate (13A) the production of mouse and human CD47-Fc assayed in the supernatant of cells transfected with the Kozak containing plasmid and (13B) an immunoblot displaying an anti-human Fc antibody reacting with the protein G-purified CD47-Fc proteins.

FIG. 14 illustrates an assay designed to quantify the binding affinity of hCD47-Fc and mCD47-Fc (not shown) to the NOD and NOR mouse versions of SIRPα.

FIGS. 15A and 15B illustrate (15A) results of binding of mCD47 to NOD and NOR SIRPα; and (15B) results of binding of hCD47 to NOD and NOR SIRPα.

FIG. 16 is a schematic showing SIRPα-CD47 mediated signaling.

FIG. 17 shows the preparation of a truncated version of the human HSC “permissive” NOD version of SIRPα (“NOD-α-cyto”) (left side).

FIG. 18 illustrates human HSC survival with NOR macrophages infected with an “empty” lentivirus (NOR-CEP “diamonds”), NOD macrophages not infected with virus (NOD uninf; filled triangles), NOD macrophages infected with “empty” lentivirus (NOD-CEP; circles), NOR macrophages infected with lentivirus containing full length NOD SIRPα (NOR-SIRP; squares), and NOR macrophages infected with lentivirus containing truncated NOD SIRPα (NOD-α-cyto; open triangles).

FIG. 19 illustrates a schematic of a study to evaluate in vivo the effect of CD47 fusion protein on HSC engraftment.

FIG. 20 illustrates that CD47 Fusion protein confers enhanced HSC engraftment in the NOD.SCID xenotransplant model.

### DETAILED DESCRIPTION

In the following description, numerous specific details are set forth to provide a thorough understanding of the invention. However, it is understood that the invention may be practiced without these specific details.

As used herein “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

As used herein, a “cultured cell” means a cell which has been maintained and/or propagated in vitro. Cultured cells include primary cultured cells and cell lines. As used herein, “culturing the cell” means providing culture conditions that are conducive to polypeptide expression. Such culturing conditions are well known in the art.

As used herein “engrafting” a stem cell, preferably an expanded hematopoietic stem cell, means placing the stem cell into an animal, e.g., by injection, wherein the stem cell persists in vivo. This can be readily measured by the ability of the hematopoietic stem cell, for example, to contribute to the ongoing blood cell formation.

As used herein “fragment” relating to a polypeptide or polynucleotide means a polypeptide or polynucleotide consisting of only a part of the intact polypeptide sequence and structure, or the nucleotide sequence and structure, of the reference gene. The polypeptide fragment can include a C-terminal deletion and/or N-terminal deletion of the native polypeptide, or can be derived from an internal portion of the molecule. Similarly, a polynucleotide fragment can include a 3′ and/or a 5′ deletion of the native polynucleotide, or can be derived from an internal portion of the molecule.

As used herein “fusion protein” refers to a composite polypeptide, i.e., a single contiguous amino acid sequence, made up of two (or more) distinct, heterologous polypeptides which are not normally fused together in a single amino acid sequence. Thus, a fusion protein may include a single amino acid sequence that contains two entirely distinct amino acid sequences or two similar or identical polypeptide sequences, provided that these sequences are not normally found together in the same configuration in a single amino acid sequence found in nature. Fusion proteins may generally be prepared using either recombinant nucleic acid methods, i.e., as a result of transcription and translation of a recombinant gene fusion product, which fusion comprises a segment encoding a polypeptide of the invention and a segment encoding a heterologous polypeptide, or by chemical synthesis methods well known in the art. Fusion proteins may also contain a linker polypeptide in between the constituent polypeptides of the fusion protein. The term “fusion construct” or “fusion protein construct” is generally meant to refer to a polynucleotide encoding a fusion protein. In one embodiment, the fusion protein is a polypeptide as described herein fused to a portion of an Ig molecule. The Ig portion of the fusion protein can include an immunoglobulin constant region, e.g. a human Cγ1 domain or a Cγ4 domain (e.g. the hinge, CH2, and CH3 regions of human IgCγ1 or human IgCγ4 (see e.g., Capon et al., U.S. Pat. Nos. 5,116,964; 5,580,756; 5,844,095, and the like). In one preferred embodiment, Ig fusion proteins include a polypeptide as described herein coupled to an immunoglobulin constant region (e.g., the Fc region).

As used herein “hematopoietic stem cell” refers to a cell of bone marrow, liver, spleen or cord blood in origin, capable of developing into any mature myeloid and/or lymphoid cell.

As used herein “isolated” with reference to a nucleic acid molecule, polypeptide, or other biomolecule, means that the nucleic acid or polypeptide has separated from the genetic environment from which the polypeptide or nucleic acid were obtained. It can also mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated nucleic acid molecules” are polypeptides or nucleic acid molecules that have been purified, partially or substantially, from a recombinant host cell or from a native source.

It is also contemplated that the peptides of the invention may exhibit the ability to modulate biological, such as intracellular, events. As used herein “modulate” refers to a stimulatory or inhibitory effect on the biological process of interest relative to the level or activity of such a process in the absence of a peptide of the invention.

The term “macrophage suppression” or “suppression of macrophages” as used herein refers to the reduction or prevention of the role, activity and/or effect of macrophages.

As used herein, the “nucleic acid molecule” means DNA molecules (e.g., a cDNA) and RNA molecules (e.g., an mRNA) and analogs of the DNA or RNA generated, e.g., by the use of nucleotide analogs. The nucleic acid molecule can be an oligonucleotide or polynucleotide and can be single-stranded or double-stranded.

As used herein “operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. Similarly, “control elements compatible with expression in a subject” are those which are capable of effecting the expression of the coding sequence in that subject.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “polypeptide” and “protein” are used interchangeably and mean proteins, protein fragments, modified proteins, amino acid sequences and synthetic amino acid sequences. The polypeptide can be glycosylated or not.

As used herein “stroma” refers to the supporting tissue or matrix of an organ, distinguishable from the functional elements of the organ or the parenchyma.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

According to one aspect, there is provided an isolated polypeptide selected from the group consisting of a polypeptide consisting of a) the amino acid sequence of SEQ ID NO. 1; b) a polypeptide consisting of a fragment of the amino acid sequence of SEQ ID NO. 1, wherein the fragment comprises at least one of residues 31, 32, 34, 37, 74, 77, 83, 84, 86, 87, 90, 91, 96, 100, 102, 114, 118, 126 of SEQ ID NO. 1; and c) one of the polypeptide in a) and b) with up to 1 amino acid insertion, deletion or substitution for every 7 amino acids in length of the polypeptide, wherein the polypeptide comprises at least one of residues 31, 32, 34, 37, 74, 77, 83, 84, 86, 87, 90, 91, 96, 100, 102, 114, 118, 126 of SEQ ID NO. 1 and binds human CD47.

According to a further aspect, there is provided an isolated polypeptide selected from the group consisting of a) a polypeptide consisting of the amino acid sequence of SEQ ID NO. 2; b) a polypeptide consisting of a fragment of the amino acid sequence of SEQ ID NO. 2; and c) one of the polypeptide in a) and b) with up to 1 amino acid insertion, deletion or substitution for every 7 amino acids in length of the polypeptide; wherein i) at least one of residues at positions 31, 32, 34, 37, 74, 77, 83, 84, 86, 87, 90, 91, 96, 100, 102, 114, 118, 126 of SEQ ID NO. 2 in the polypeptide is replaced with corresponding residues 31, 32, 34, 37, 74, 77, 83, 84, 86, 87, 90, 91, 96, 100, 102, 114, 118, 126 of SEQ ID NO. 1; or ii) at least one of residues 129 and 130 of SEQ ID NO. 2 in the polypeptide is deleted.

According to a further aspect, there is provided an isolated polypeptide selected from the group consisting of a) a polypeptide consisting of an amino acid sequence selected from the group consisting of SEQ ID NOS. 4-13; b) a polypeptide consisting of a fragment of an amino acid sequence selected from the group consisting of SEQ ID NOS. 4-13; and c) one of the polypeptide in a) and b) with up to 1 amino acid insertion, deletion or substitution for every 7 amino acids in length of the polypeptide, wherein the polypeptide binds human CD47.

According to a further aspect there is provided a polypeptide capable of binding to human Sirpα; and antibodies to human Sirpα. Preferably, the polypeptide is the extracellular domain of human CD47 fused to the Fc portion of IgG.

In some embodiments, the polypeptide is the fragment thereof and comprises at least 3 consecutive amino acids in at least one of a region between residues 50-57, 63-71, 74-80, 88-92, 95-100, 103-109, 114-125 or 128-141, inclusive of SEQ ID NO. 1, between residues 50-57, 63-71, 74-80, 88-92, 95-100, 103-109, 114-125 or 128-143, inclusive of SEQ ID NO. 2, between residues 24-31, 37-45, 48-54, 62-66, 69-74, 77-83, 88-99 or 102-116, inclusive, of any one of SEQ ID NOs. 4, 7, 8, 9 and 12; or between residues 24-31, 37-45, 48-54, 62-66, 69-74, 77-83, 88-99 or 102-115, inclusive, of SEQ ID NOs. 5, 6, 10, 11 and 13, as the case may be.

In some embodiments, the amino acid insertion, deletion or substitution is a conservative amino acid substitution.

In a preferred embodiment, the polypeptide binds human CD47.

In a preferred embodiment, the polypeptide binds human SIRPα.

In some embodiments, in increasing preferability, the polypeptide is the fragment and is between 6 and 30 amino acids in length, between 8 and 28 amino acids in length, between 10 and 26 amino acids in length, between 12 and 24 amino acids in length and between 14 and 22 amino acids in length.

The polypeptides described herein may be fused to a second polypeptide that is preferably the Fc portion of IgG.

According to one preferred embodiment, there is provided a polypeptide comprising the extracellular domain of CD47 fused to the Fc portion of IgG, preferably for increasing hematopoietic stem cell engraftment in a human.

According to a further aspect, there is provided a pharmaceutical composition comprising a polypeptide described herein and a pharmaceutically acceptable carrier.

According to a further aspect, there is provided a method for increasing hematopoietic stem cell engraftment in a mammal comprising administering to the mammal a therapeutically effective amount of a polypeptide described herein. Preferably, the increased hematopoietic stem cell engraftment results from suppression of macrophages.

According to a further aspect, there is provided a use of a polypeptide described herein for increasing hematopoietic stem cell engraftment in a mammal or in the preparation of a medicament for increasing hematopoietic stem cell engraftment in a mammal.

According to a further aspect, there is provided a polypeptide described herein for increasing hematopoietic stem cell engraftment in a mammal.

According to a further aspect, there is provided an isolated nucleic acid comprising a sequence that encodes a polypeptide described herein.

According to a further aspect, there is provided an expression vector comprising a nucleic acid described herein, preferably comprising a Kozak consensus.

According to a further aspect, there is provided a cultured cell comprising a vector described herein.

According to a further aspect, there is provided a method of producing a polypeptide comprising culturing a cell described herein under conditions permitting expression of the polypeptide.

According to a further aspect, there is provided a method for increasing hematopoietic stem cell engraftment in a human comprising modulating the interaction between human Sirpα and human CD47. Preferably, the interaction between human Sirpα and human CD47 is modulated by administering a therapeutically effective amount of at least one of a) a polypeptide capable of binding to the extracellular domain of human Sirpα; b) antibodies to human Sirpα ; c) a polypeptide capable of binding to the extracellular domain of human CD47; and d) antibodies to human CD47. Correspondingly, there is also provided use of the foregoing for increasing hematopoietic stem cell engraftment in a human or for preparing medicament for the same. Preferably, the increased hematopoietic stem cell engraftment results from suppression of macrophages.

In one embodiment, the polypeptide capable of binding to the extracellular domain of human Sirpα comprises soluble human CD47, or a fragment thereof, preferably the extracellular domain of CD47. Preferably, the soluble human CD47, or a fragment thereof, is fused to a second protein. In another embodiment, the polypeptide capable of binding to the extracellular domain of human CD47 comprises soluble human Sirpα, or a fragment thereof, preferably the extracellular domain of Sirpα. Preferably, the soluble human Sirpα, or a fragment thereof, is fused to a second protein. In preferred embodiments, the second protein is the Fc portion of IgG.

According to a further aspect, there is provided a method of identifying a compound that increases hematopoietic stem cell engraftment in a human comprising a) contacting at least one of the extracellular domain of human Sirpα and human CD47 with at least one test compound; b) determining the at least one test compound as binding to the at least one of human Sirpα and human CD47; c) contacting the test compound with human hematopoietic cells in a stromal environment; and d) determining whether hematopoietic stem cell engraftment is increased in the presence of the test compound.

According to a further aspect, there is provided a method of determining genetic polymorphisms in humans affecting hematopoietic stem cell engraftment comprising a) sequencing the Sirpα gene from a plurality of humans having undergone hematopoietic transplantation; b) determining nucleotide differences in the Sirpα gene within the plurality of humans; and c) correlating the nucleotide differences with hematopoietic stem cell engraftment to determine relevant polymorphisms. Preferably, the nucleotide differences result in amino acid differences.

According to a further aspect, there is provided a method of determining likelihood of hematopoietic stem cell engraftment in a recipient comprising a) sequencing the Sirpα gene from the recipient; and b) determining whether the relevant polymorphisms exist.

Preferably, the relevant polymorphisms result in an amino acid difference that is preferably at least one of (a) replacement of at least one of residues at positions 31, 32, 34, 37, 74, 77, 83, 84, 86, 87, 90, 91, 96, 100, 102, 114, 118, 126 of SEQ ID NO. 2 with corresponding residues 31, 32, 34, 37, 74, 77, 83, 84, 86, 87, 90, 91, 96, 100, 102, 114, 118, 126 of SEQ ID NO. 1; or (b) deletion of at least one of residues 129 and 130 of SEQ ID NO. 2.

The advantages of the present invention are further illustrated by the following examples. The examples and their particular details set forth herein are presented for illustration only and should not be construed as a limitation on the claims of the present invention.

### EXAMPLES

### Materials & Methods

### Mice

The CB17-scid/scid (SCID), NOD/LtSz-Prdkc^{sc/sc} (NOD.SCID) (Shultz, L. D. et al. (1995) J Immunol 154, 180-91), C57BL/6 (B6), C3H, ICR, and BALB/c mice were bred and maintained under sterile conditions at the Ontario Cancer Institute (Toronto, Canada). Rag-2^{-/-} (RAG-2), RAG-2.Prf^{-/-}, and RAG-2.β₂m^{-/-} mice, all on B6 background, were obtained from GenPharm International (Mountain View, Calif.). NOD, NOD.SCID, NOD.NOR-Idd13, NOD.NOR-Idd4, NOD.NOR-Idd5, NOD.NOR-Idd9, NOR.NOD-Idd13, and B6.NOD-Idd13 mice were maintained in either specific pathogen-free or barrier conditions at the Hospital for Sick Children (Toronto, Ontario).

All congenic mice were generated with marker-directed selection of breeders from an initial intercross of NOD/Jsd and NOR/Lt progenitors. The NOD and NOR genomes are approximately 88% identical by descent, and the differential portions of their genomes are distributed on chromosomes 1, 2, 4, 5, 7, 10, 11, 12, 14 and 18. Breeders were screened with microsatellite markers for all genetic loci that differ between NOD and NOR, such that the founders of all congenic lines were homozygous for background alleles. The microsatellite markers used to define the congenic mice used in this study are listed in Table 1.

TABLE 1
Microsatellite markers used to map congenic mice.

| Markers used to define congenic intervals | | | | | | |
|---|---|---|---|---|---|---|
| | | | | | | SEQ |
| | | | Position | | | ID |
| Locus | Marker | Chr. | (Mb) | Primer 1 | Primer 2 | NO. |
| IBD | D1Gul117 | 1 | 72614682 | GAACATAGGTGCAC | TGATGCTGGAGGTT | 19/20 |
| | | | | ACTCACATA | GTCCT | |
| Idd5 | D1Ngul46 | 1 | 72932895 | GATGAAAGAGATAA | TGGTTATTAAAGAG | 21/22 |
| | | | | GACAGATGTTAGAT | CTGTTTTACATG | |
| | | | | A | | |
| Idd5 | D1Mit77 | 1 | 74154726 | AAGTTGGAACTCTG | GTGTCTTCAATGCA | 23/24 |
| | | | | CAGGACA | GCACGT | |
| Idd5 | D1Mit132 | 1 | 77603656 | TATTGTTTATGGAA | CATCTCTGAAGGAA | 25/26 |
| | | | | ATTGGACCC | AAAGTGCA | |
| Idd5 | D1Mit216 | 1 | 80318888 | GGGAGACAACAAAT | AGAGGTGGGTCCTG | 27/28 |
| | | | | AATCATATTGC | GAAACT | |
| Idd5 | D1Mit8 | 1 | 83489415 | CTGAAAATCGTCCC | CAGGAGCATGAAA | 29/30 |
| | | | | TTGACC | TGGGGAT | |
| Idd5 | D1Mit440 | 1 | 88698913 | TCCACACAAGGTGT | GCTCAGGTGACCTC | 31/32 |
| | | | | CCTCTG | CAAAAC | |
| Idd5 | Ramp1 | 1 | 91094432 | ATCACCACTGTGGG | CAGTAGAGGCCAA | 33/34 |
| | | | | CATTCTGAAC | GGGCATCAGAATC | |
| IBD | D1Mit84 | 1 | 91834941 | TGTCTCCCCAAAGT | GTGATGCAGGAGTT | 35/36 |
| | | | | AGCAGG | TCTGCA | |
| IBD | D2Jyh439 | 2 | 118988748 | GGCAGATTTATCGC | GTGCGCCACCAATG | 37/38 |
| | | | | TGTGAG | C | |
| Idd13 | D2Jyh443 | 2 | 119047301 | CTTCAGTGCTGTATC | CTCCCACTCCCATT | 39/40 |
| | | | | CACTGC | AATTCTC | |
| Idd13 | D2Mit17 | 2 | 122563069 | AGGCAATTACAAGG | CACCCATCTCCCTC | 41/42 |
| | | | | CCTGG | AGTCAT | |
| Idd13 | D2Mit256 | 2 | 126365634 | GTAGAGACTTGGTG | ATCTCTGCTGACAA | 43/44 |
| | | | | GGTTTTGG | AGAACAGTG | |
| Idd13 | D2Mit447 | 2 | 128530442 | CATGTGCCATGGTA | TGGCTTGTTTCAGA | 45/46 |
| | | | | CAAAGC | TAGCACA | |
| Idd13 | D2Mit338 | 2 | 130578309 | TCACCAGCCTGAAA | TCTGGGTACAATCC | 47/48 |
| | | | | ACACTG | TTAGTCCTG | |
| Idd13 | D2Mit490 | 2 | 138644398 | GCCAGATACAATTT | GTGGGACGCAATAT | 49/50 |
| | | | | TCCCTCA | TGGC | |
| Idd13 | D2Mit452 | 2 | 162450015 | TCCCACATTTCTGGC | CACATGTGCATTTA | 51/52 |
| | | | | ATACA | AGCATGC | |
| IBD | D2Mit51 | 2 | 163213661 | GTGAGGGGTCAATG | GGCTCAGTTGTAAG | 53/54 |
| | | | | CCAC | CACAAGG | |
| IBD | D4Mit331 | 4 | 102248682 | CCTAACCCTCCCCA | AAAGATCTGGATTC | 55/56 |
| | | | | CACC | AAATCCTCC | |
| Idd9 | D4Mit31 | 4 | 105457920 | ACGAGTTGTCCTCT | AGCCAGAGCAAAC | 57/58 |
| | | | | GATCAACA | ACCAACT | |
| Idd9 | D4Mit308 | 4 | 122531464 | TATGGATCCACTCT | CAAAGTCTCCTCCA | 59/60 |
| | | | | CCAGAAA | AGGCTG | |
| Idd9 | D4Mit204 | 4 | 131880763 | CTGCTGCAGCGATT | TCAGGCACCTAAGT | 61/62 |
| | | | | CTCTC | ACATGTGC | |
| Idd9 | D4Mit251 | 4 | 135384963 | AAAAATCGTTCTTT | TTTAAAAGGGTTTC | 63/64 |
| | | | | GACTTCTACATG | TTTATCCTGTG | |
| Idd9 | D4Mit259 | 4 | 142770937 | TTTTCTTAGAACATA | GGGGAAGGTGAAA | 65/66 |
| | | | | CCTTGCTTGG | ATTTAAAGG | |
| Idd9 | D4Mit310 | 4 | 146582497 | TCTCCACGTGTGTG | TGAAAGCACTCTGC | 67/68 |
| | | | | CCTTAG | AGACTCA | |
| Idd9 | D4Mit189 | 4 | 146994411 | CACTACACGGGGCT | AAGATCCAACACCC | 69/70 |
| | | | | GAGATT | TTTTTGG | |
| IBD | D4Ngul7383 | 4 | 147772405 | GGCAGCTTTGTGCT | ATGGATGTTTACCT | 71/72 |
| | | | | TTAAAA | GGCGT | |
| Idd4 | D11Mit217 | 11 | 37214368 | ACTGGAAAATATGT | AAATGGGATTCTGC | 73/74 |
| | | | | TTTAAACCTCTG | AAAAACC | |
| Idd4 | D11Mit310 | 11 | 53814488 | GTGCACTTTCCATG | GAGTAGAAAGAGA | 75/76 |
| | | | | CCTGTA | CAGAGAAAGACAC | |
| | | | | | A | |
| Idd4 | D11Mit90 | 11 | 70112196 | TCTCCAGCCCCTTCA | TGCCAAACACCCAT | 77/78 |
| | | | | TTATG | GAGAC | |
| Idd4 | D11Jyh1081 | 11 | 80495333 | TCCTTGGTGAGTCC | GGCTAGGCTCTGGA | 79/80 |
| | | | | ACATCT | AGACA | |
| IBD | D11Mit42 | 11 | 112795824 | ACTAGCCATATGGT | GTAGCAGGGCTGTG | 81/82 |
| | | | | TTCTGATGG | AGCTTT | |
| Markers used to screen for background alleles in congenic mice | | | | | | |
| | | | position | | | SEQ ID |
| locus | marker | Chr. | (cM) | primer1 | primer2 | NO. |
| non- | D1Mit430 | 1 | 6.6 | TATTAATGTTGAA | CTTTAATCATCTCT | 83/84 |
| Idd | | | | GCCAGAAGCC | GTGGCAAGG | |
| non- | D1Mit122 | 1 | 20.4 | TGAAGCAAGAATT | AGGACATACCTAGT | 85/86 |
| Idd | | | | CAAGAAGAGG | AAGGCTTTGTC | |
| non- | D5Mit11 | 5 | 17.5 | GATCTTCCTACCTT | CATGATTTTATTTG | 87/88 |
| Idd | | | | CTTACCCAC | GGGGG | |
| non- | D7Mitl24 | 7 | 37.2 | GTCAGACATTGGC | GGTTTGTGCGCTCT | 89/90 |
| Idd | | | | TTAGGATCC | CTCTCT | |
| non- | D10Mit86 | 10 | 12 | TTTGCCTGTAACAA | TTGAGGCTATCAGT | 91/92 |
| Idd | | | | GCCAGA | TTAAAATCCC | |
| non- | D11Mit62 | 11 | 2.2 | GAATAACCCATGT | CTCTGGACTTGTGT | 93/94 |
| Idd | | | | TTATATCGGTG | TCTATGCC | |
| non- | D12Mit76 | 12 | 38.3 | CCTCCTAATAGTGC | CCAGCAATCACGAG | 95/96 |
| Idd | | | | CATTCCC | TCTTCC | |
| non- | D14Mit194 | 14 | 52.5 | GACCAAAGTCATA | TTAATTGCAAGTAA | 97/98 |
| Idd | | | | TCTATAACACC | CACAATGAGTAGG | |
| non- | D18Mit4 | 18 | 37.2 | ACTGTTGCTGGGG | CCAAGTTCAAAGCT | 99/100 |
| Idd | | | | AATGG | GCTGG | |

IBD =identical by descent;
non-Idd = regions of non-identity between NOD and NOR outside the four Idd regions

NOD.NOR-Idd13.SCID mice were generated from an intercross of NOD.NOR-Idd13 mice to NOD.SCID mice, followed by brother-sister matings screened by marker assisted genotyping until homozygosity was achieved at both Idd13 and SCID loci.

For microsatellite genotyping, genomic DNA was prepared from 0.6-0.8 cm tail snips that were digested in a solution of proteinase K and digestion buffer (AutoGen AG00122) overnight at 55° C. The crude digests were diluted and heated to inactivate the proteinase K. PCR was performed with oligonucleotide primers modified at their 5′ ends with fluorescent tags. PCR conditions were the same for all microsatellite primers: 10 cycles of 30 s at 94° C., 30 s at 50° C., and 1 min at 72° C., followed by 35 cycles of 30 s at 94° C., 30 s at 55° C., and 1 min at 72° C. in 1.5 mM MgCl₂. PCR amplicons were detected with the ABI 3730XL sequencer and alleles were called with the aid of the GeneMapper software version 3.0 (Applied Biosystems, Foster City, Calif.).

For LTC on MS-5 murine stromal cells, non-irradiated MS-5 cells were seeded into 96 well tissue culture plates (2000 cells/well) as described in Gan, O. I. et al. (1999) Exp Hematol 27, 1097-106. Three days later, murine peritoneal macrophages were seeded at doses of 20-20,000 cells/well with 5 replicates per dose. The next day, 2000 human Lin⁻ CB cells per well were added in 200 μl human LTBMC medium without hydrocortisone. Cultures were maintained at 33° C. (or 37° C. for gene transfer experiments) with weekly half-medium change. After 3-4 weeks, cells were trypsinized and half of the cells from each well were plated into progenitor assays as described.

### Human-Specific Hematopoietic Progenitor Cell Assay

Seven thousand five hundred cells from T25 flasks, or half well content from 96-well plates, were plated in 1 ml methylcellulose cultures under conditions that are selective for human cells as previously described in Larochelle, A. et al. (1996) Nat Med 2, 1329-37. The cultures were incubated for 14 days at 37° C. and the total number of colonies produced by colony-forming cells (CFC) was counted. Cells from T25 flasks were plated in triplicate.

### Murine Macrophage Preparation

Murine peritoneal macrophages were obtained by peritoneal levage with 10 ml of αMEM with 10% FCS. BM-derived macrophages were obtained by flushing femurs, tibias, and iliac crests with 10 ml of DMEM supplemented with 10% FCS, 10 mM HEPES (pH 7.0), 50 nM 2-mercaptoethanol, 2 mM glutamine, 1× nonessential amino acids, and 1% penicillin/streptomycin (“complete DMEM”). BM cells were plated at a concentration of 1×10⁶ cells/ml in complete DMEM containing 10 ng/ml recombinant mouse GM-CSF (R&D Systems, Minneapolis, Minn.). Medium was changed with fresh complete DMEM with GM-CSF every other day, and macrophages were harvested by gentle scraping after 7 days.

### Real Time PCR Analysis of Idd13 Candidate Genes

Expression of genes within the 0.96 Mb critical Idd13 interval was analyzed by PCR amplification of cDNA prepared from BM stroma and purified macrophages. Real time PCR was performed using an ABI/PRIZM 7900 HT cycler (Applied Biosystems) under the following conditions: 95° C. for 15 s and 59° C. for 1 min for 40 cycles, with addition of SYBR Green buffer (Applied Biosystems). Serial dilutions of a plasmid containing the gene sequence were used as a reference for standard curve calculation. The fluorescence thresholds and corresponding copy number were calculated using SDS 2.0 software (Applied Biosystems). Reactions were performed in triplicate, and transcript quantity was normalized to β-actin.

### Molecular Genetic Analysis of Murine Sirpα

The coding region of Sirpα was sequenced from NOD, NOR and BALB/c strains. cDNA was prepared from BM-derived macrophages and used as a PCR template under the following conditions: 30 s at 94° C., 30 s at 60° C., and 1 min at 68° C. in 2 mM MgSO₄for 30 cycles using High-Fidelity Taq polymerase (Invitrogen Life Technologies, Carlsbad, Calif.). The untranslated (UTR) region of Sirpα was also amplified and sequenced in NOD and NOR strains, using genomic DNA as a template and the same PCR conditions. Sequencing primers were designed using Primer Express 1.5 software (Applied Biosystems) and are listed in Table 2. PCR products were purified according the manufacturer's instructions (Qiagen, Valencia, Calif.) and sequenced on both strands at the Center for Applied Genomics, Hospital for Sick Children, Toronto, Canada (TCAG). B6, BALB/c, and 129/Sv mouse Sirpα sequence files were obtained from EnsEMBL and NCBI databases. Sequence files were aligned and analyzed with Lasergene software (DNASTAR, Madison, Wis.).

TABLE 2
Primers used to PCR amplify and sequence coding and
untranslated regions of murine and human SIRPα

| | | | SEQ |
|---|---|---|---|
| | | | ID |
| | Forward Primer | Reverse Primer | NO. |
| Mouse | | | |
| SirPα- | AGTCCACCTTAAGAGGACCAA | TGTACAGAAACAGGACGCGGA | 101/102 |
| 1 | GTAGC | | |
| Sirpα- | TCTCCCTCCTTGCTCTGCAG | TACCTCGCAGATGACCTTAGAA | 103/104 |
| 2 | | TTAA | |
| Sirpα- | CCTAGTGGAAAGAATGTCTCCT | CATATTCTGTGTGGTTGTTAGGC | 105/106 |
| 3 | ACAACA | TCA | |
| Sirpα- | CCAGGTACAGTCTTTGATCCAG | CCAGGGAGTCTCTGCTGGTCTA | 107/108 |
| 4 | GA | | |
| Sirpα- | CATCTGTGGAAGAACTACACC | TACCTCAGAGAGCTGGCCGAGT | 109/110 |
| 5 | AGAAGTC | | |
| Sirpα- | CCAATGCTGACCTAATGTTGGC | CATCTATACACCCGTGTGTGTAT | 111/112 |
| 6 | | ACACA | |
| Sirpα- | AACTTGTCTTTGTCCGGGCC | GGCACAGTTCATCCTCACCC | 113/114 |
| 7 | | | |
| Human | | | |
| Sirpα | TAGAATACAGGCTCATGTTGC | GCCTTCAGCAAATAGCATGACG | 115/116 |
| | AGGT | T | |

### Molecular Genetic Analysis of Human SIRPα

Thirty-seven unrelated individuals from the HapMap collection were chosen for sequencing based on genotype variation at two markers flanking the IgV domain encoded in exon 3 (rs6075340 and rs611968). DNA samples were obtained from TCAG and exon 3 was amplified by PCR using High-Fidelity Taq polymerase (Invitrogen Life Technologies). The resulting amplicons were cloned and exon 3 regions from 2 to 4 independent clones per individual were sequenced at TCAG on both strands using primers flanking the plasmid multiple cloning site (Table 2). Sequences were aligned using Lasergene software.

### Biochemical Analysis of SIRPα

BM-derived macrophages were washed with ice-cold PBS and then lysed in buffer (20 mM Tris-HCl pH 7.6, 140 mM NaCl, 1 mM EDTA, 1% NP-40) containing 5 mM NaF, aprotinin (10 μg/ml) and 1 mM sodium vanadate. The lysates were centrifuged at 10 000 g for 15 min at 4° C. and the resulting supernatants subjected to immunoblot analysis as follows: proteins (20 μg per well) were electrophoresed in 10% sodium dodecyl sulphate (SDS)-polyacrylamide gels and transferred onto 0.2 μM nitrocellulose membranes (Bio-Rad, Hercules, Calif.). Western blots were analyzed using anti-SIRPα antibody (Stressgen, Victoria, BC, Canada) and developed with the HRP-ECL system (Amersham Biosciences, Piscataway, N.J.). N-linked oligosaccharides were removed from SIRPα by boiling cell lysates for 5 minutes in 0.5% SDS and 1% βmercaptoethanol to denature the glycoproteins, and then incubating with N-glycosidase F (40 units/ml, Roche Applied Science, Indianapolis, Ind.) for 12 hours at 37° C. For immunoprecipitation, 250 μg of BM-derived macrophage lysate was added to either hCD47-Fc (10 μg/ml) or the control human IgG Fc (10 μg/ml) with 50 μl of protein G MicroBeads (Miltenyi Biotec, Auburn, Calif.). Following a 60-minute incubation on ice, the immune complexes were collected on a μMACS separator following manufacturer's instructions (Miltenyi Biotec, Auburn, Calif.) and eluted from the column with SDS-PAGE gel loading buffer.

### Sirpα Lentiviral Infection Protocol

NOD Sirpα cDNA was cloned downstream of an E1Fα promoter in a third-generation lentiviral vector backbone containing a reporter Gfp gene driven by the human PGK promoter (SIRP). The control vector (CEP) contained only Gfp. Vector construction was confirmed by sequencing. Viruses pseudotyped with the vesicular stomatitis virus G protein were generated by transient transfection of 293T cells, as described in Guenechea, G. et al. (2000) Mol Ther 1, 566-73, and concentrated by ultracentrifugation. The functional titers of SIRP and CEP virus as determined by infection of HeLa cells were 2.1-5.1×10⁸ and 4-7.6×10⁸ infectious particles per ml, respectively. Virus was added to flasks of NOR BM-derived macrophages after 5 days of culture at a multiplicity of infection of 20 to 25, and infected macrophages were harvested on day 12. Gene transfer efficiency into macrophages was 51-88% as assessed by GFP fluorescence using flow cytometry. Infected macrophages were stained with PE-conjugated anti-mouse CD11b mAb (Beckman Coulter, Fullerton, Calif.) and sorted on a Dako Cytomation MoFlo (Fort Collins, Colo.) to obtain GFP⁺ CD11b⁺ cells. Uninfected control macrophages were stained and sorted to obtain CD11b⁺ cells. Purified macrophages (purity 97-99%) were used for further experiments.

### Soluble Human CD47-Fc Preparation

293T cells were transfected with pcDNA-CD47Fc containing the human CD47 extracellular domain fused to human IgG1 Fc directed by the CMV promoter (Latour, S. et al. (2001) J Immunol 167, 2547-54) using a Superfect Transfection Kit (Qiagen) and selected with 100 μg/ml Zeocin (Invitrogen, Carlsbad, Calif.). Cell lines producing soluble human CD47-Fc (hCD47-Fc) were identified by immunoblot analysis of culture supernatants. hCD47-Fc protein was purified on Protein G-Sepharose (Pierce, Rockford, Ill.) and covalently conjugated to biotin for use in flow cytometric analyses.

### Flow Cytometry

Flow cytometric analysis was performed using a FACSCalibur (BD, Franklin Lakes, N.J.). For analysis of human cell engraftment in mice, cells harvested from murine BM were stained with phycoerythrin (PE) cyanine5-conjugated anti-human CD45 antibody (Beckman Coulter). For analysis of cell surface expression of SIRPα and binding of hCD47-Fc, 1×10⁶ murine BM leukocytes were stained with purified anti-SIRPα mAb P84 (BD) with Alexa 633-conjugated goat anti-rat (Molecular Probes, Carlsbad, Calif.), FITC-conjugated anti-CD11b mAb (SWRI, San Antonio, Tex.), and either biotinylated hCD47-Fc with avidin-APC or a control human IgG Fc fragment with biotinylated anti-human IgG mAb and avidin-APC. Viable leukocytes were selected by exclusion of propidium iodide. Isotype controls were mouse or rat IgG conjugated to the appropriate fluorochrome. GFP fluorescence was detected in FL1 calibrated to the fluorescein isothiocyanate emission profile.

### Statistical Analysis

Groups were compared using one-way ANOVA and all pairwise multiple comparisons were done using Dunn's method when differences were found. For gene transfer experiments, CEP and SIRP conditions were compared using the Wilcoxon rank test.

### Protein Synthesis

The human CD47 IgV domain cDNA sequence was cloned in-frame upstream of a human IgG Fc cDNA sequence, inserted into appropriate expression vectors, transfected into 293F cells, and the resulting fusion protein (hCD47-Fc) purified from culture supernatant by affinity chromatography on protein G beads. Mouse CD47-Fc (mCD47-Fc) fusion protein was prepared using the same strategy.

Assay of Binding Affinity of hCD47 and mCD47 to mSIRPα Expressed on Mouse Macrophages

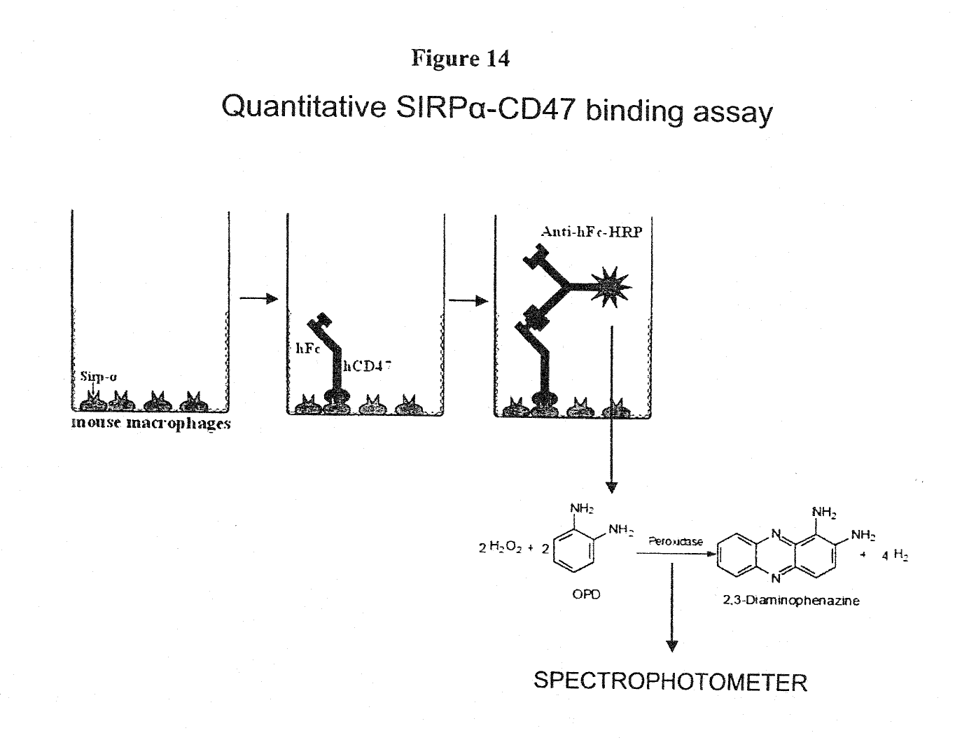

FIG. 14 is a schematic representation of an assay designed to quantify the binding affinity of hCD47-Fc and mCD47-Fc (not shown) to the NOD and NOR mouse versions of SIRPα. Macrophages obtained from NOD or NOR mice were allowed to adhere to tissue culture plates for 2 hours then washed with buffered saline. Graded amounts of purified hCD47-Fc or mCD47-Fc fusion protein was added to the wells, allowed to bind at 37° C. for 20 minutes, and the wells washed extensively with buffered saline. A commercially available anti-human Fc antibody covalently conjugated to horseradish peroxidase (HRP) was added and allowed to bind, and the plates washed extensively with buffered saline. The quantity of bound hCD47 was evaluated by colorimetric assay following HRP reaction with hydrogen peroxide and peroxidase treatment using a spectrophotometer.

### Example 1

Immune-Deficient Mice with NOD Strain Background Support Human Hematopoietic Cell Engraftment In Vivo

Several groups including our own have shown that NOD mice homozygous for the Prkdc^{scid} (SCID) mutation permit superior human hematopoietic cell engraftment compared to SCID mice on other strain backgrounds, including CB17 (Greiner, D. L. et al. (1995) Am J Pathol 146, 888-902; Larochelle, A. et al. (1996) Nat Med 2, 1329-37). The molecular and cellular basis for this strain difference in xenograft efficacy is not known. However, NOD.SCID mice have a high incidence of spontaneous thymic lymphoma that limits their usefulness for long-term experiments. Mice carrying null alleles of the recombinase activating gene 2 (Rag-2) on the C57BL/6 (B6) background have similar blockade in T and B cell development to CB17.SCID mice but do not display spontaneous lymphoid malignancies (Shinkai, Y. et al. (1992) Cell 68, 855-67). Rag-2^{-/-} (RAG-2) mice also carrying homozygous null alleles in β₂ microglobulin (RAG-2.β₂m^{-/-}) or perforin (RAG-2.Prf^{-/-}) possess additional defects in natural killer (NK) cell function (Zijlstra, M. et al. (1990) Nature 344, 742-6; Kagi, D. et al. (1994) Nature 369, 31-7). We therefore tested several other immunodeficient strains of mice for their ability to support human hematopoiesis.

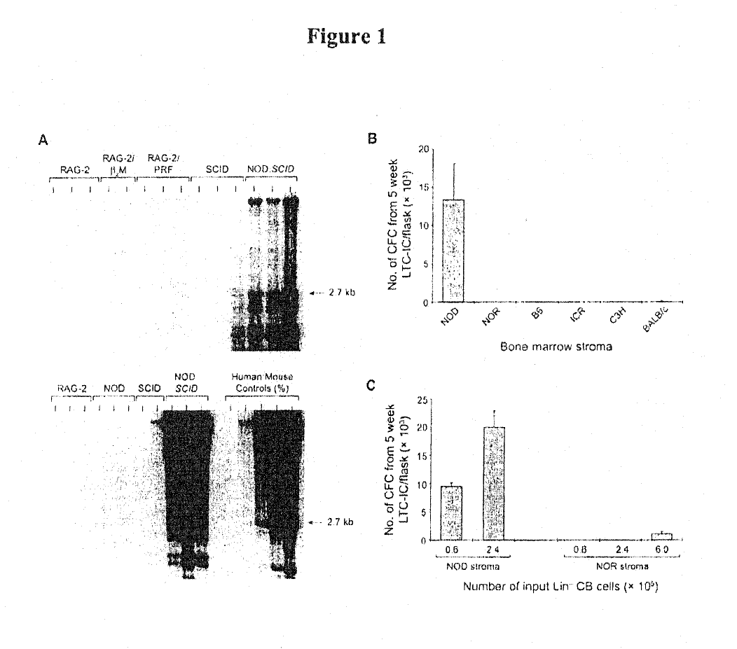

Human BM and CB cells were transplanted into mice from each strain and engraftment was assessed by Southern blot analysis (FIG. 1A). Consistently high levels of engraftment were detected in NOD.SCID mice, whereas CB17.SCID mice showed a lower frequency and level of engraftment, as previously reported (Larochelle, A. et al. (1996) Nat Med 2, 1329-37). As expected, no engraftment was detected in immune-competent NOD recipients. Surprisingly, despite their profound immune-deficiency, engraftment failed in RAG-2, RAG-2.Prf^{-/-}, and RAG-2.β₂m^{-/-} mice on the B6 background. In contrast, NOD.Rag-1^{-/-} mice have been shown to support high levels of human hematopoietic cell engraftment (Shultz, L. D. et al. (2000) J Immunol 164, 2496-507). These observations suggest that additional factors other than immune-deficiency in the mouse strain background and/or host BM microenvironment dramatically impact hematopoietic engraftment in the xenotransplant setting.

### Example 2

### Support of Human Long-Term Culture Initiating Cells Requires NOD Alleles on Chromosome 2

Long-term culture initiating cells (LTC-IC) are the most primitive human hematopoietic cells that can be assayed in vitro (Wang, J. C. Y. et al. “Normal and leukemic human stem cells assayed in immune-deficient mice” in: Hematopoiesis—A Developmental Approach (ed. Zon, L. I.) 99-118 (Oxford University Press, New York, USA, 2001), and serve as a surrogate measure of human hematopoiesis under controlled microenvironmental conditions. LTC-IC are quantified based on their ability to generate colony-forming cells (CFC) after 5 weeks of stromal culture. To characterize strain differences in the support of human hematopoiesis, we compared the ability of BM stromal cells from NOD/Jsd (NOD) and other strains to support LTC-IC. Stromal layers from NOD mice supported human CFC production for 5 weeks of culture, whereas BM stroma from all of the other strains did not support human LTC-IC following inoculation with equivalent numbers of Lin⁻ CB cells (FIG. 1B). NOD stroma was supportive even at low input numbers of Lin⁻ CB cells (0.6×10⁵; FIG. 1C). We examined NOR/Lt (NOR) mice in this assay because they are a recombinant inbred strain 88% identical by descent to NOD, differing only at four Idd loci (Idd4, Idd5, Idd9, and Idd13) (Prochazka, M. et al. (1992) Diabetes 41, 98-106). In contrast to NOD, NOR cultures supported LTC-IC only when seeded with 10-fold greater numbers of Lin⁻ CB cells (FIG. 1C), and resulted in small-sized CFC. These data suggest that the unique support of primitive human hematopoiesis observed in NOD mice in vitro and in vivo is controlled by regions of genetic non-identity between NOD and NOR mice.

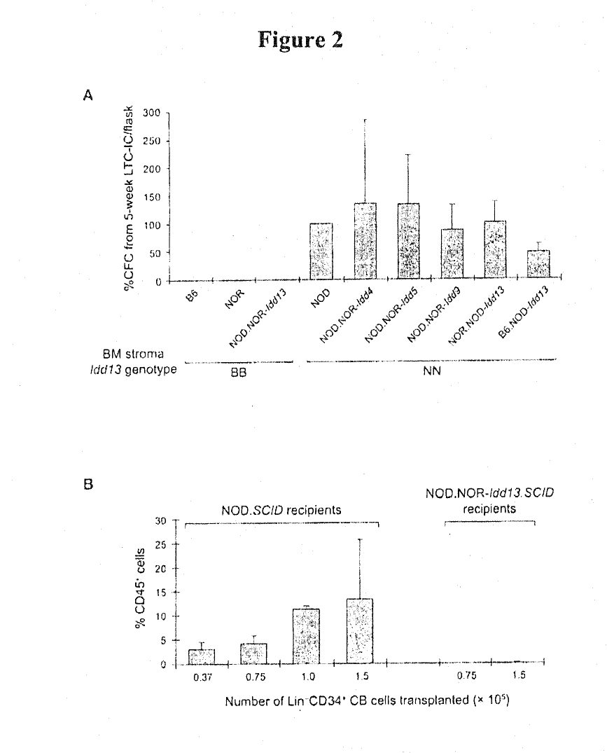

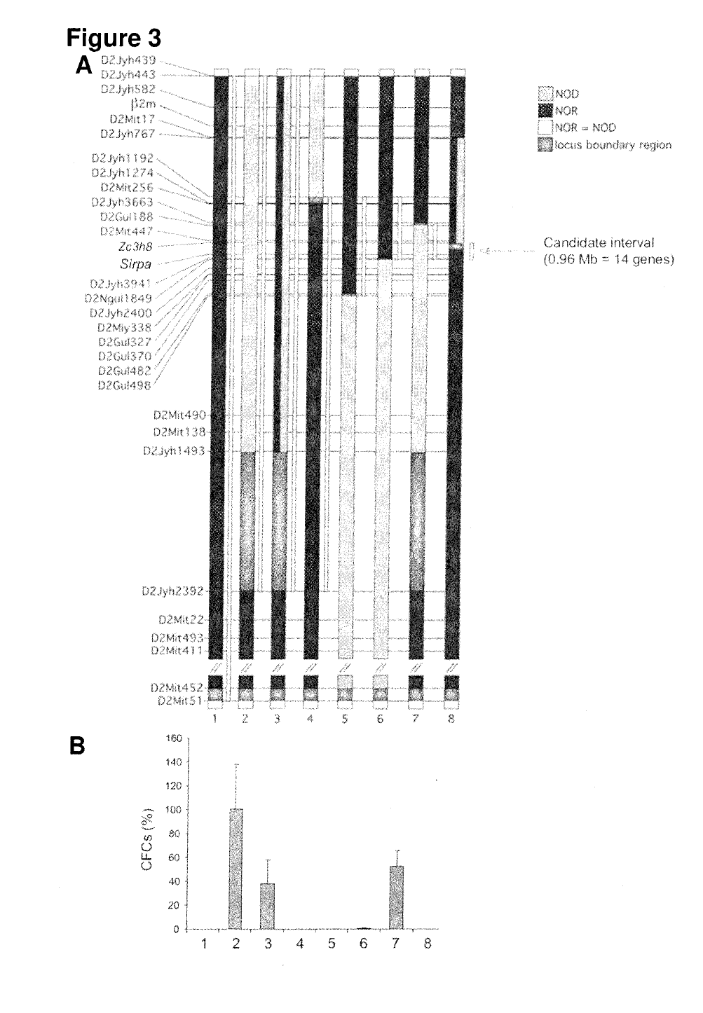

To map the genetic loci that conferred the superior ability of the NOD strain to support human hematopoiesis, stromal cells from NOD and NOR parental mice, a series of NOD and NOR-Idd congenic strains, and B6.NOD-Idd13 mice were tested in chimeric LTC (FIG. 2A). Neither the B6 or NOR parental strains were able to support development of human LTC-IC compared to NOD stroma. NOD mice congenic for NOR alleles at Idd4, Idd5, and Idd9 displayed equivalent LTC-IC outcomes to NOD parental animals. In contrast, stromal cells isolated from NOD.NOR-Idd13 mice (Idd13-BB genotype) were unable to support human LTC-IC whereas both B6 and NOR mice congenic for NOD Idd13 (Idd13-NN genotype; B6.NOD-Idd13 and NOR.NOD-Idd13) were supportive. Thus, the NOD-derived Idd13 locus on chromosome 2 (Serreze, D. V. et al. (1994) J. Exp. Med. 180, 1553-1558) conferred dramatically enhanced capacity of BM stromal cells to support human LTC-IC in vitro.

### Example 3

### Idd13 Genotype Determines Support of Human Hematopoietic Stem Cell Engraftment In Vivo

To test whether the Idd13 locus controls support of human hematopoietic cells capable of repopulation in vivo (termed SCID-repopulating cells, SRC), we generated an immune-deficient NOD congenic strain homozygous for NOR-derived Idd13 (NOD.NOR-Idd13-SCID). Sublethally irradiated NOD.SCID and NOD.NOR-Idd13-SCID mice were transplanted intravenously with Lin⁻ CB cells (equivalent to 0.37-1.5×10⁵ CD34⁺ cells) and human CD45⁺ cell engraftment was assessed after 6-7 weeks by flow cytometry (FIG. 2B). As expected, NOD.SCID mice supported human engraftment over the entire CB cell dose range. In contrast, no human cell engraftment was detected in NOD.NOR-Idd13-SCID mice. These results confirm the in vitro LTC-IC data and establish that NOD alleles at the Idd13 locus confer support of human hematopoiesis in vivo.

### Example 4

### Fine Mapping of Candidate Genes at Idd13 Regulating Support of Human Hematopoiesis

To refine the location of gene(s) at Idd13 that regulated support of human hematopoiesis, we used a mapping strategy involving generation of Idd13 congenic strains on both the NOD or NOR background (FIG. 3A). Stromal cells from these congenic animals were tested in chimeric LTC assays (FIGS. 3A and B). Interestingly stromal cells from Idd13 heterozygous mice (Idd13-BN) supported human LTC-IC, however the number of CFC generated was consistently ˜50% of that seen in NOD cultures (FIGS. 3A and B; third bars from left). These data were consistent with a dominant effect of NOD Idd13 alleles on support of human LTC-IC. By repeating these analyses on eight novel Idd13 congenic strains on both the NOD and NOR backgrounds, the support of human LTC-IC phenotype was resolved to a 960 kb region (FIGS. 3A and B), defined by a single nucleotide polymorphism in Fliz1 and the microsatellite D2Ngul1849 (Table 3). Of 15 genes in the interval, eight had functional annotation. Expression of seven of the eight genes in BM stroma and macrophages of NOD and NOR strains was assessed using real time PCR (Table 4). Sirpα (also called Shps-1 and Ptpns1) was the only gene expressed in BM stroma that showed coding sequence polymorphism between NOD and NOR and, thus, was selected as the most promising candidate for further study.

TABLE 3
Idd13 candidate interval supporting human LTC-IC.

| | Start | Genomic | | | | | SEQ ID |
|---|---|---|---|---|---|---|---|
| Chr | position | marker | EnsEMBL gene ID | Gene symbol | Primer 1 | Primer 2 | NO. |
| 2 | 128675293 | Fliz1 | ENSMUSG00000027387 | Zc3hdc8 | | | |
| 2 | 128716430 | | ENSMUSG00000042851 | ZCC6_MOUSE | | | |
| 2 | 128744547 | D2Ngul1725 | | | GCCACTAC | CCTGTCTTG | 117/118 |
| | | | | | CATCCGAT | AAAACCCA | |
| | | | | | TCTT | AAA | |
| 2 | 128815007 | | ENSMUSG00000027394 | Ttl | | | |
| 2 | 128850037 | | ENSMUSG00000027395 | Rpo1-2 | | | |
| 2 | 128878840 | | ENSMUG00000037938 | Chchd5 | | | |
| 2 | 128917969 | | ENSMUSG00000043404 | — | | | |
| 2 | 128947823 | | ENSMUSG00000027397 | Slc20a1 | | | |
| 2 | 128967992 | | ENSMUSG00000056738 | A730036I17Rik | | | |
| 2 | 129017904 | | ENSMUSG00000048327 | 2610318C08Rik | | | |
| 2 | 129048633 | | ENSMUSG00000027399 | Il1α | | | |
| 2 | 129113614 | | ENSMUSG00000027398 | Il1β | | | |
| 2 | 129118920 | | ENSMUSG00000056635 | NM_177848 | | | |
| 2 | 129207382 | | ENSMUSG00000043727 | F830045P16Rik | | | |
| 2 | 129342601 | | ENSMUSG00000037902 | Sirpα = Ptpns1 | | | |
| 2 | 129435589 | | ENSMUSG00000027400 | NDDB_MOUSE | | | |
| 2 | 129530898 | D2Jyh3941 | | | AATGACTT | TTTTATGCC | 119/120 |
| | | | | | GTTTGCCA | CTAGGAGC | |
| | | | | | AGGA | CTC | |
| 2 | 129549775 | | ENSMUSG | Stk35 | | | |
| | | | [symbol] | | | | |
| 2 | 129635018 | D2Ngul1849 | | | ACTCTCTG | CCTCACCCT | 121/122 |
| | | | | | AGTAGCCT | TACTTGGC | |
| | | | | | GCCCC | ACC | |

[symbol]

indicates data missing or illegible when filed

The candidate interval supporting human LTC-IC is bounded by Fliz1 and D2Ngul1849, (see FIG. 3A) defining a 0.96 Mb interval on chromosome 2 containing 15 genes. A minimal candidate locus was defined by microsatellite markers D2Ngul1725 and D2Jyh3941 and contained 13 genes. All genomic positions and gene attributes are based on EnsEMBL version 26.33b.1. Fliz1 primers were designed to detect a polymorphism between NOD and NOR strains in the 5′ UTR.

TABLE 4
Expression of annotated genes in Idd13 candidate interval.

| | | | | | Mean copy |
|---|---|---|---|---|---|
| | | | | | number normalized |
| Gene | | | | Mean copy | to β-actin ± |
| symbol | EnsEMBL ID | Genotype | Cell type | number ± SD | SD (×10⁻³) |
| Fliz1 | ENSMUSG00000027387 | NOR | stroma | 2180 ± 177 | 2.04 ± 0.17 |
| | | NOR.NOD-Idd13 | stroma | 2107 ± 257 | 1.97 ± 0.24 |
| | | NOR | macrophage | UD | UD |
| | | NOR.NOD-Idd13 | macrophage | UD | UD |
| Rpo1-2 | ENSMUSG00000027395 | NOR | stroma | 9955 ± 1836 | 7.66 ± 1.41 |
| | | NOR.NOD-Idd13 | stroma | 4938 ± 1234 | 6.86 ± 1.7.1 |
| | | NOR | macrophage | 399 ± 23 | 0.40 ± 0.02 |
| | | NOR.NOD-Idd13 | macrophage | 225 ± 39 | 0.54 ± 0.09 |
| Chchd5 | ENSMUSG00000037938 | NOR | stroma | UD | UD |
| | | NOR.NOD-Idd13 | stroma | UD | UD |
| | | NOR | macrophage | UD | UD |
| | | NOR.NOD-Idd13 | macrophage | UD | UD |
| Slc20a1 | ENSMUSG00000027397 | NOR | stroma | 13762 ± 467 | 23.80 ± 0.81 |
| | | NOR.NOD-Idd13 | stroma | 19926 ± 1767 | 22.39 ± 1.99 |
| | | NOR | macrophage | ND | ND |
| | | NOR.NOD-Idd13 | macrophage | ND | ND |
| Il1α | ENSMUSG00000027399 | NOR | stroma | 12104 ± 1206 | 10.09 ± 1.01 |
| | | NOR.NOD-Idd13 | stroma | 11193 ± 3229 | 9.33 ± 2.69 |
| | | NOR | macrophage | 2321338 ± 571008 | 1304.12 ± 320.79 |
| | | NOR.NOD-Idd13 | macrophage | 4303725 ± 191113 | 1198.81 ± 53.24 |
| Il1β | ENSMUSG00000027398 | NOR | stroma | 19813 ± 1701 | 17.08 ± 1.40 |
| | | NOR.NOD-Idd13 | stroma | 12324 ± 3185 | 15.40 ± 3.90 |
| | | NOR | macrophage | 9167234 ± 122794 | 7051.72 ± 94.46 |
| | | NOR.NOD-Idd13 | macrophage | 8730824 ± 608149 | 4595.18 ± 320.08 |
| Stk35 | ENSMUSG00000037885 | NOR | stroma | 221 ± 16 | 0.44 ± 0.44 |
| | | NOR.NOD-Idd13 | stroma | 400 ± 15 | 0.57 ± 0.02 |
| | | NOR | macrophage | ND | ND |
| | | NOR.NOD-Idd13 | macrophage | ND | ND |

UD = below limit of detection
ND = not done
See Materials and Methods for description of real time PCR

### Example 5

### Strain-Dependent Sequence and Biochemical Variation in Idd13 Candidate Gene Sirpα

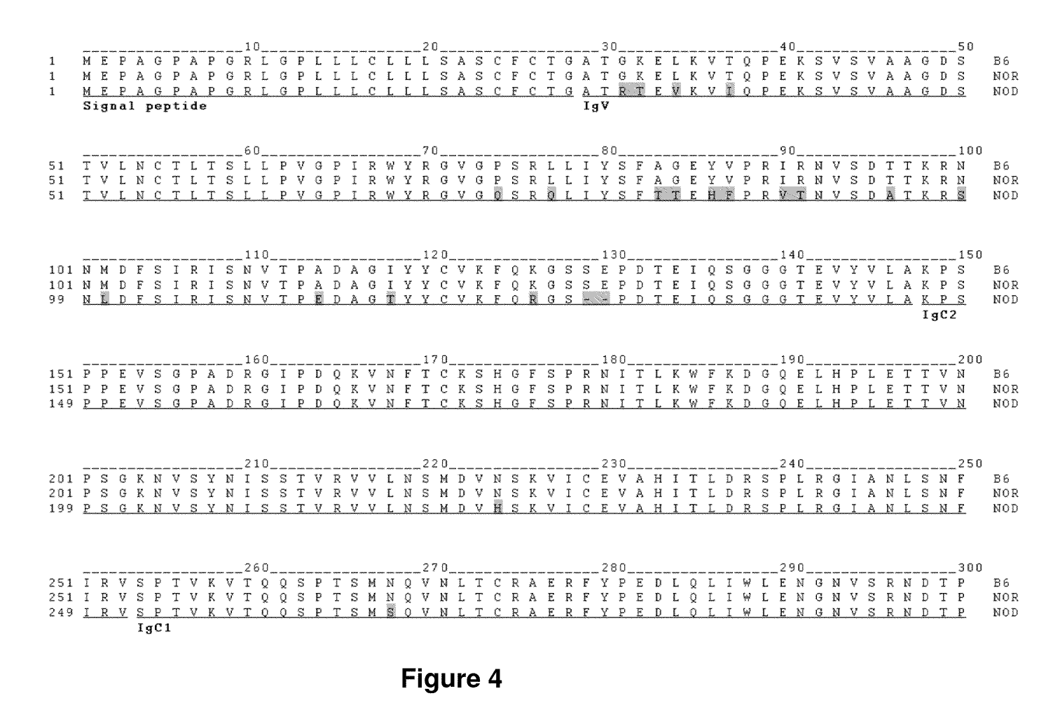

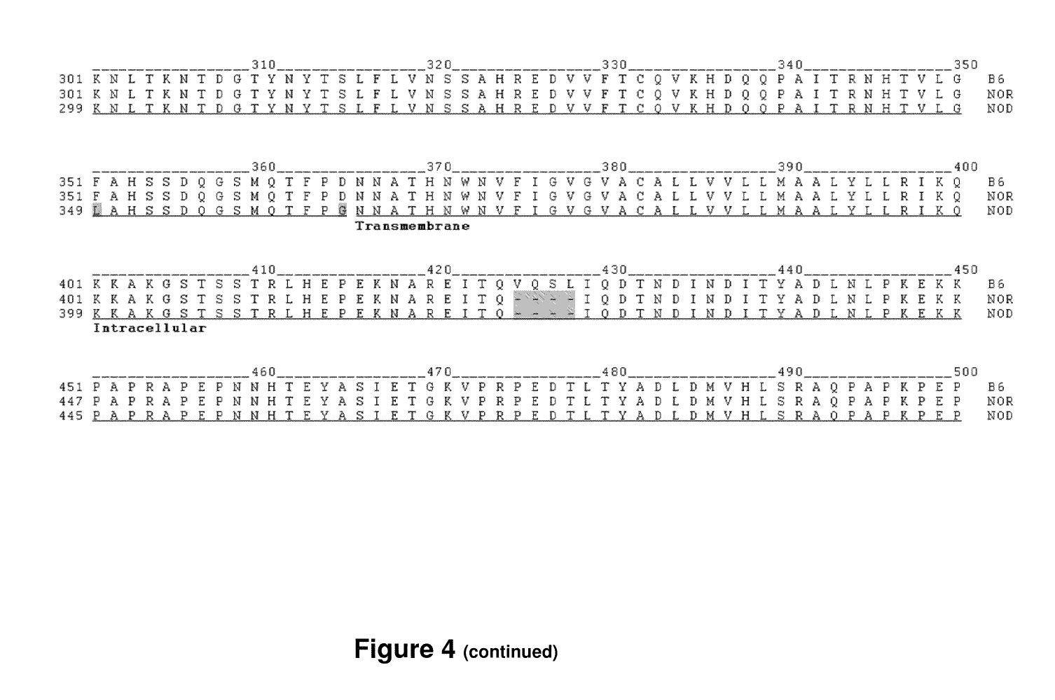

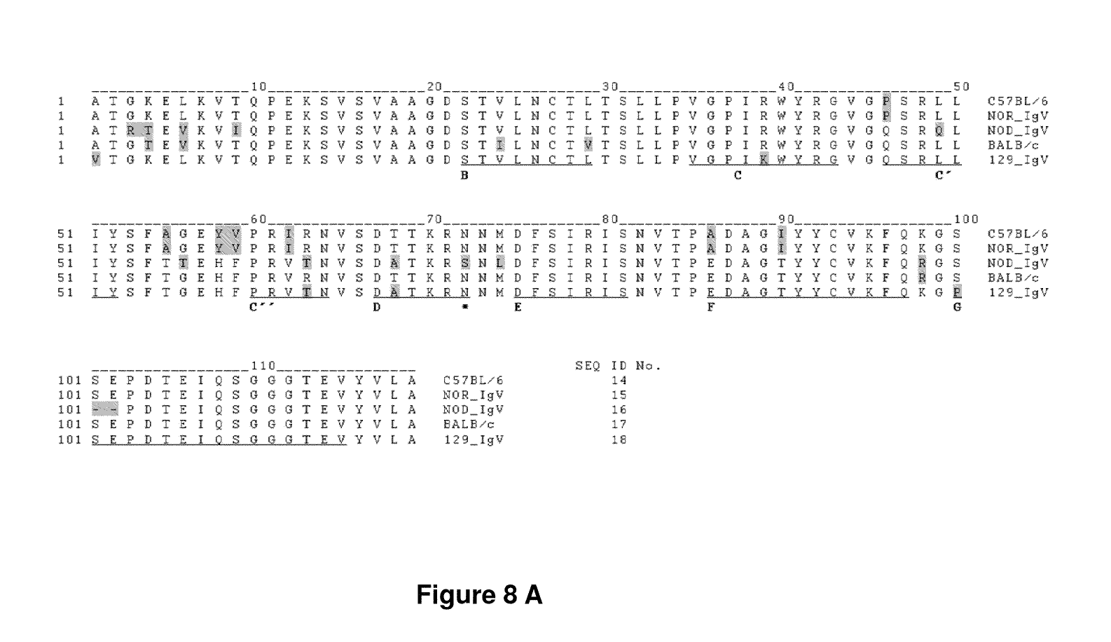

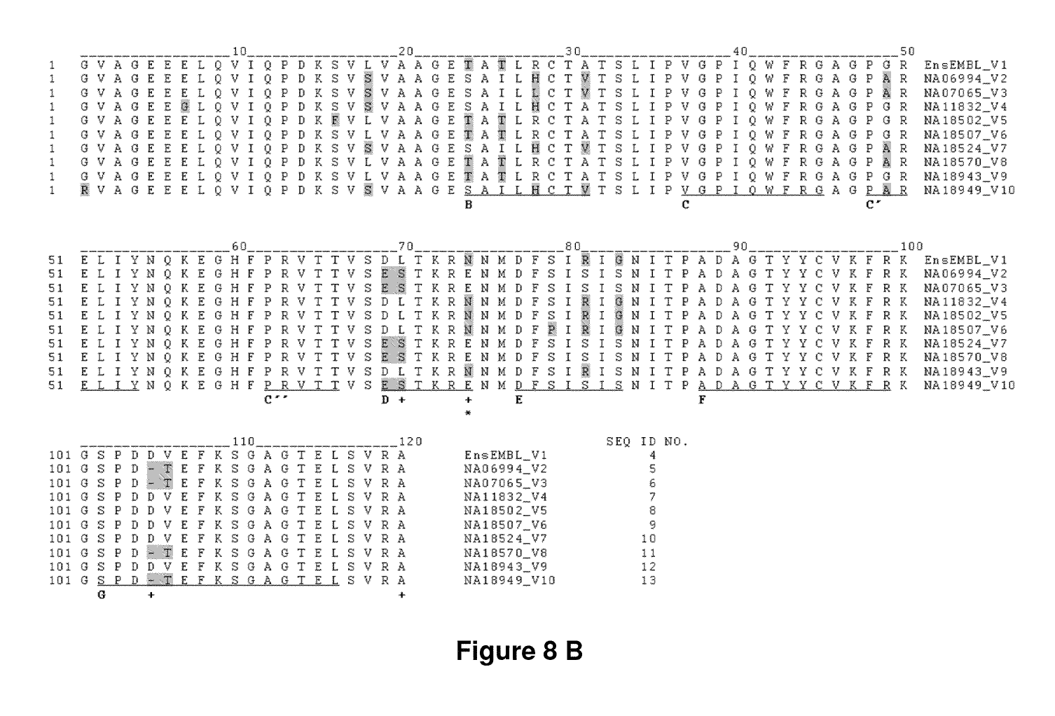

Signal regulatory protein α (SIRPα) is an Ig-superfamily transmembrane protein with intracellular docking sites for two Src homology domain-containing protein tyrosine phosphatases SHP-1 and SHP-2 (Barclay, A. N. & Brown, M. H. (2006) Nat Rev Immunol 6, 457-64). Sirpα is abundantly expressed in BM stroma and hematopoietic cells (data not shown). We used PCR amplification and bi-directional sequencing of cDNA from BM-derived macrophages to identify variations in the coding regions of NOD, NOR and BALB/c mice. The NOR sequence is identical to B6 with the exception of 4 additional amino acids in the B6-derived cytoplasmic domain (FIG. 4) that likely reflects variation between two splice donor sites in exon 7 (Sano, S. et al. (1999) Biochem J 344 Pt 3, 667-75). Comparison of the Sirpα coding sequence between NOD and NOR revealed 24 amino acid differences, 20 of these in the extracellular IgV-like domain of molecule where the NOD sequence displays 18 substitutions and two deletions compared to NOR and B6 (FIG. 4 and FIG. 8A). This observed variation in the Sirpα the N-terminal IgV-like domain between NOD and NOR or B6 is more extensive than that previously reported in this region amongst the B6, BALB/c and 129/Sv strains (Sano, S. et al. (1999) Biochem J 344 Pt 3, 667-75).

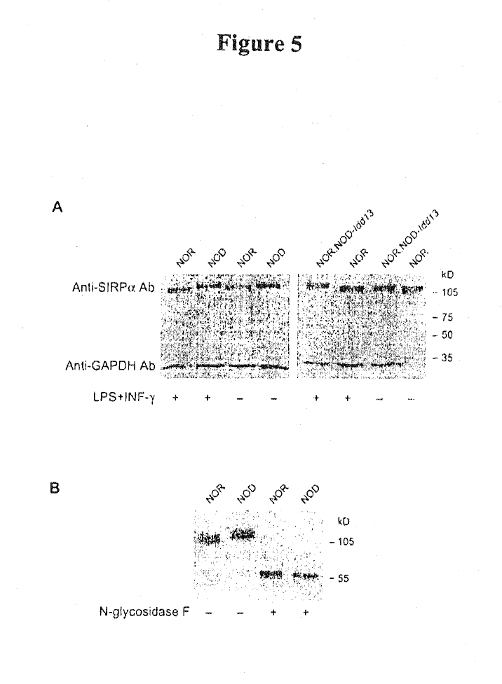

To assess the consequences of SIRPα sequence variation between NOD and NOR mice, protein lysates were prepared from BM-derived macrophages, and SIRPα expression was examined by immunoblotting with polyclonal antibodies directed against the cytoplasmic domain which is sequence identical in NOD and NOR mice (FIG. 4). No quantitative strain differences in SIRPα expression were detected under the conditions examined. Electrophoretic mobility of the NOD protein was slightly less than that of the NOR protein (FIG. 5A). SIRPα is glycosylated (van den Nieuwenhof, I. M. et al. (2001) J Cell Sci 114, 1321-9), so we examined the possibility that strain differences in the apparent molecular weight reflected differential glycosylation. Protein lysates from BM-derived macrophages were treated with N-glycosidase F and examined by immunoblotting (FIG. 5B). Following removal of N-linked glycans, SIRPα from both strains migrated faster, with roughly equal mobility. These results suggest that the extensive polymorphism we detected in the extracellular portion of the NOD and NOR proteins results in differential glycosylation. Thus, the amino acid sequence and post-translational modifications dissimilarities between NOD and NOR strains are consistent with Sirpα being the Idd13 gene conferring strain-specific differences in human hematopoietic support.

### Example 6

### Murine Macrophages Display Strain-Specific Variation in Support of Human LTC-IC

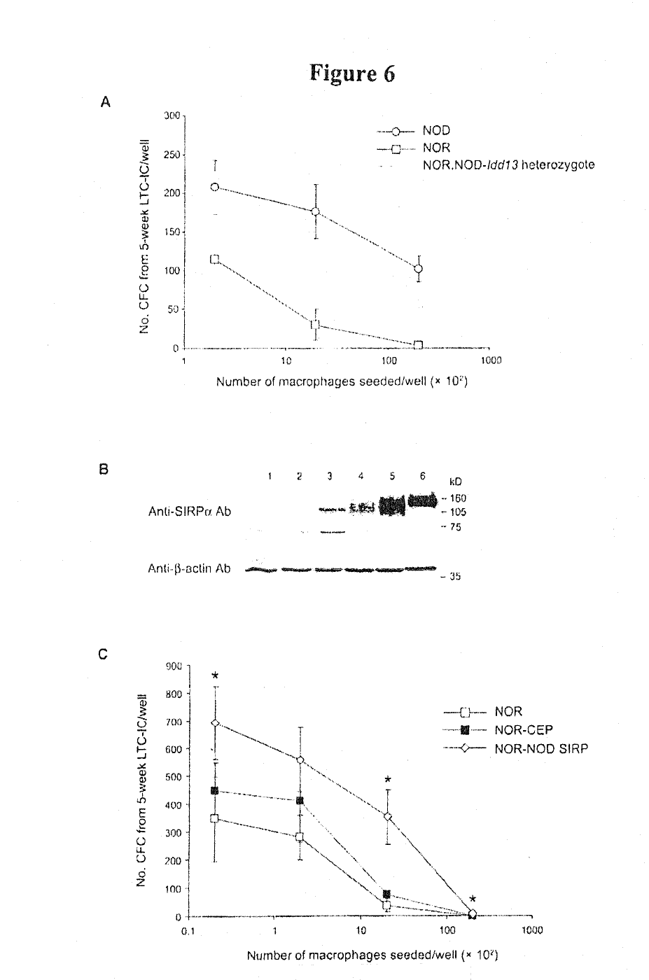

Murine SIRPα is most abundantly expressed in neurons and myeloid cells (Veillette, A. et al. (1998) J Biol Chem 273, 22719-28) with evidence of differentially glycosylated forms in heart, liver, kidney and other tissues (Sano, S. et al. (1999) Biochem J 344 Pt 3, 667-75). SIRPα ligation regulates multiple macrophage functions including inhibition of phagocytosis (Oldenborg, P. A. et al. (2001) J Exp Med 193, 855-62; Blazar, B. R. et al. (2001) J Exp Med 194, 541-9) and TNFα production (Smith, R. E. et al. (2003) Blood 102, 2532-40), and increasing nitric oxide production (Alblas, J. et al. (2005) Mol Cell Biol 25, 7181-92). We and others have shown that a human CD122⁺ cell subset distinct from NK cells, likely macrophages, may play a role in host resistance to human hematopoietic engraftment in the xenotransplantation setting (McKenzie, J. L. et al. (2005) Blood 106, 1259-61; Shultz, L. D. et al. (2003) Exp Hematol 31, 551-8). In addition, macrophages are a component of hematopoietically supportive BM stromal cultures (Hauser, S. P. et al. (1995) J Histochem Cytochem 43, 371-9). Based on these observations, we tested whether macrophages conferred the strain-specific differences in human hematopoietic support we observed in chimeric LTC-IC assays. Peritoneal macrophages from NOD, NOR, and NOR congenic mice heterozygous at the Idd13 locus (NOR.BN-Idd13) were harvested and added to human LTC-IC cultures employing MS-5 stromal cells, a murine cell line that supports human hematopoiesis (Issaad, C. et al. (1993) Blood 81, 2916-24). Murine macrophages were seeded onto established MS-5 cultures at doses of 200, 2,000 and 20,000 cells per well, followed 24 hours later by addition of 2,000 Lin⁻ CB cells. Macrophages of all strains had a dose-dependent effect on the number of CFC generated after 3 to 4 weeks in LTC (FIG. 6A). NOR macrophages exerted greater suppression of human LTC at all cell doses than NOD macrophages; at 2,000 and 20,000 macrophages/well, the strain difference was significant (p<0.008). NOR.BN-Idd13 had an intermediate effect between NOR and NOD macrophages consistent with our previous observations of a (co)-dominant effect of Sirpα alleles on support of human hematopoiesis. Replicate experiments performed with BM-derived macrophages showed similar results (data not shown). These findings demonstrate a Idd13 genotype-specific, dose-dependent suppressive effect of murine macrophages on human hematopoiesis in vitro.

### Example 7

### Sirpα Confers Differential Capacity of NOD Macrophages to Support Human Hematopoiesis

To directly test whether the NOD Sirpα variant confers the observed strain-specific effects of macrophage-mediated suppression of human hematopoiesis, we constructed lentivirus vectors expressing NOD-derived Sirpα plus Gfp (SIRP), or Gfp alone (CEP). Infection of Jurkat cells or NOR BM-derived macrophages with SIRP resulted in high level expression of SIRPα protein, which in macrophages exhibited NOD-type mobility on Western blot analysis suggesting that it was appropriately glycosylated in these cells (FIG. 6B) To assess the effect of Sirpα in chimeric LTC, NOR BM-derived macrophages were infected with SIRP or control CEP lentivirus prior to seeding on MS-5 stromal cultures. Human Lin⁻ CB cells were added the next day and generation of CFC was assessed 25 to 31 days later. Cell dose effects were again evident in this setting where seeding 20,000 macrophages/well suppressed human hematopoiesis regardless of Sirpα genotype. However, expression of NOD-derived SIRPα by NOR macrophages resulted in substantially greater human CFC support compared to either non-manipulated or CEP-infected NOR macrophages (FIG. 6C and data not shown). Results from these gain-of-function experiments confirm that SIRPα confers strain-specific, macrophage-mediated effects in chimeric LTC, providing direct support that this gene is responsible for the Idd13 locus effect on the support of human hematopoesis both in vitro and in vivo.

### Example 8

### Allelic Variation in Murine Sirpα Confers Differential Binding to Human CD47

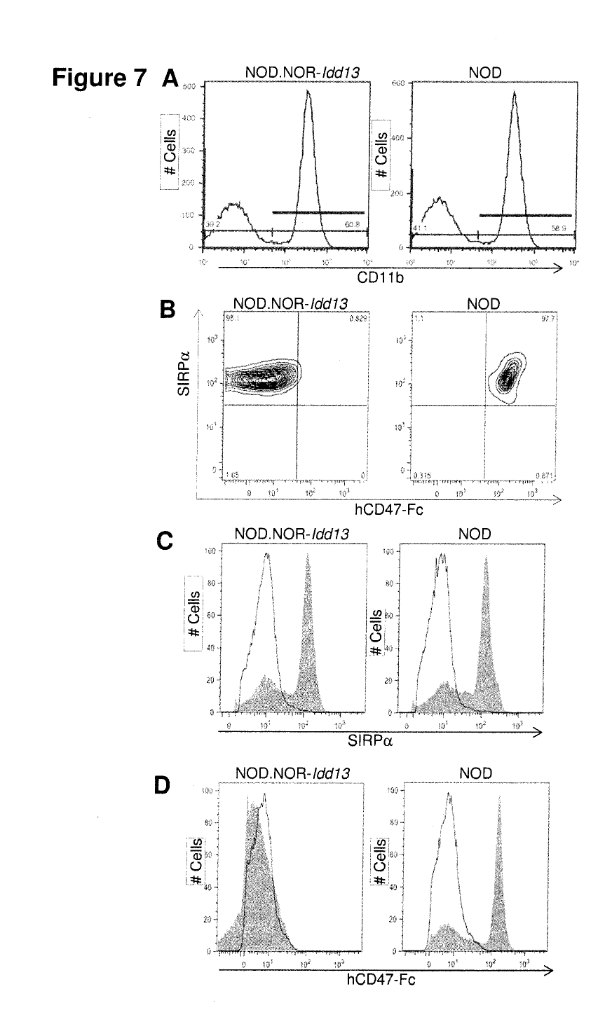

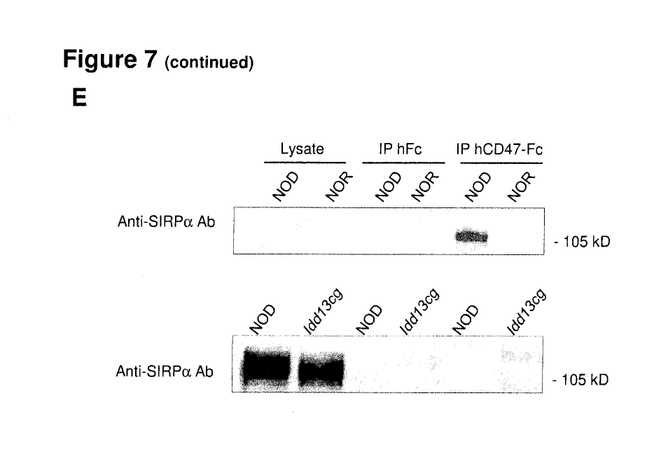

The cellular ligand of SIRPα is the ubiquitously expressed integrin-associated protein CD47, also a member of the Ig superfamily (Seiffert, M. et al. (1999) Blood 94, 3633-43). CD47 binds to the extracellular IgV-like domain of SIRPα (Seiffert, M. et al. (2001) Blood 97, 2741-9; Vernon-Wilson, E. F. et al. (2000) Eur J Immunol 30, 2130-7). Previous studies have shown that human SIRPα displays cross-species binding to porcine CD47, but not to CD47 from mouse (B6), rat or cow (Subramanian, S. et al. (2006) Blood 107, 2548-56). Having identified 20 amino acid differences in the IgV-like domain between the NOD and NOR alleles of SIRPα (FIG. 4), we asked whether these variations conferred differential binding of murine SIRPα to human CD47. BM-derived macrophages from NOD and NOD.NOR-Idd13 mice had similar proportions of CD11b⁺ cells (FIG. 7A), and these cells expressed equivalent amounts of SIRPα (FIGS. 7B and C). However, the human CD47 extracellular domain fused to human IgG1 Fc (hCD47-Fc) (Latour, S. et al. (2001) J Immunol 167, 2547-54) showed far greater binding to NOD compared to NOD.NOR-Idd13 macrophages (FIGS. 7B and D), demonstrating Idd13-genotype-dependent interaction between murine SIRPα and human CD47. NOD, NOR and NOD.NOR-Idd13 macrophage extracts contained equal amounts of SIRPα, but immunoprecipitation of SIRPα by hCD47-Fc was neglible from both NOR and NOD.NOR-Idd13 lysates, in contrast to the efficient recovery from NOD lysates (FIG. 7E). Taken together, these data indicate that NOD-derived SIRPα displays far greater reactivity with human CD47 than does the NOR (or B6)-derived protein, providing a molecular mechanism for the enhanced support of human hematopoesis in NOD.SCID mice.

### Example 9

### Protein Sequence Alignments of Murine and Human SIRPαIgV Domains and Sirpα Polymorphisms Within Human Populations

Above, we have used a positional genetics approach combined with functional assays of hematopoiesis to identify a Sirpα as an important regulator of interactions between human stem and progenitor cells and the BM microenvironment in vitro and in vivo. We show that expression on murine macrophages of a SIRPα protein variant capable of binding human CD47 is both necessary and sufficient for support of human hematopoiesis in chimeric LTC. These findings implicate macrophages as important mediators of engraftment following hematopoietic stem cell transplantation.

The superior human hematopoietic engraftment achieved in NOD.SCID mice compared to other immune-deficient mouse strains suggests that additional host strain-specific factors affect hematopoietic engraftment. We found a robust genetic association between the Idd13 locus in murine macrophages and support of human hematopoiesis in chimeric cultures and in vivo. Direct gain-of-function studies using lentiviral gene transfer confirm the in vitro LTC-IC data and implicate Sirpα as the gene responsible for the Idd13 locus effect. In the hematopoietic system, SIRPα is primarily expressed on myeloid cells and contains cytoplasmic immunoreceptor tyrosine-based inhibition motifs (ITIM) which mediate inhibitory signals leading to reduced phagocytosis by macrophages, inhibition of neutrophil migration, and attenuated production of the inflammatory cytokine TNFα (reviewed in Barclay, A. N. & Brown, M. H. (2006) Nat Rev Immunol 6, 457-64). CD47, the only known cellular ligand of SIRPα, is ubiquitously expressed and modulates multiple cellular actions on hematopoietic cells including platelet activation, migration, and adhesion, leukocyte adhesion and cytokine production, and T cell responsiveness (reviewed in Brown, E. J. & Frazier, W. A. (2001) Trends Cell Biol 11, 130-5). Both SIRPα and CD47 are immunoglobulin superfamily members and their interaction is mediated through their respective IgV-like domains (Seiffert, M. et al. (2001) Blood 97, 2741-9). We identified 20 amino acid differences in the IgV-like domain between NOD and NOR SIRPα, as well as dissimilar glycosylation between the two proteins, suggesting that differential interaction with human CD47 could underlie the strong effect of Sirpα variation on support of human hematopoiesis in vitro and in vivo. This mechanism is supported by our biochemical and flow cytometric analyses demonstrating enhanced binding to human CD47 by the NOD-derived SIRPα compared to the NOR-derived protein.

CD47-SIRPα interaction has been implicated in regulation of phagocytosis-mediated clearance of red blood cells (Oldenborg, P. A. et al. (2000) Science 288, 2051-4) and leukocytes (Gardai, S. J. et al. (2005) Cell 123, 321-34), wherein expression of CD47 on the hematopoietic cells serves as a “marker of self”, inhibiting phagocytosis by macrophages expressing SIRPα. A similar mechanism was suggested to modulate phagocytosis of a porcine lymphoblastoid cell line (LCL) by human macrophages recovered from liver allografts, based on in vitro observations that phagocytosis was attenuated by pre-incubation of the human macrophages with human CD47-Fc, or by ectopic expression of human CD47 on LCL transfectants (Ide, K. et al. (2007) Proc Natl Acad Sci USA 104, 5062-6). Our evidence extends these prior observations by demonstrating that genetic variation in the Sirpα IgV domain impacts the degree of CD47 interaction and can exert an “all or nothing” effect on engraftment of human HSC, linking the previous findings to an in vivo transplantation setting in which outcome is modulated by host macrophages. Importantly, without being bound to any theory, macrophage-mediated phagocytosis may not be the sole or primary effector mechanism underlying hematopoietic support in vitro or in vivo, as we observed a gradual disappearance of human hematopoietic cells in NOR chimeric cultures (data not shown), an observation not readily consistent with rapid phagocytosis-based clearance. Moreover, macrophages from all mouse strains displayed a dose-dependent suppressive effect on the growth of human LTC-ICs, suggesting that SIRPα signals inhibit macrophage secretion of inflammatory cytokines such as TNFα, as previously shown for macrophages stimulated by pathogen products (Smith, R. E. et al. (2003) Blood 102, 2532-40). In addition to SIRPα effects on macrophages, SIRPα-CD47 binding may activate CD47-induced signalling pathways within the human hematopoietic cells. Thus, any number of the pleiotropic effects of SIRPα-CD47 interactions may influence human HSC survival and engraftment.

Our observations in the xenotransplantation setting may have broader implications for clinical HSC transplantation. There is a large body of evidence demonstrating that the observed behavior of primitive hematopoietic stem/progenitor cells transplanted into NOD.SCID mice is predictive of the clinical setting (Wang, J. C. Y. et al. “Normal and leukemic human stem cells assayed in immune-deficient mice” in: Hematopoiesis—A Developmental Approach (ed. Zon, L. I.) 99-118 (Oxford University Press, New York, USA, 2001).

To further explore whether the functional variations we have observed in the murine system are relevant in humans, we investigated SIRPα polymorphism in human populations. We sequenced the SIRPα IgV domain from 37 unrelated normal Caucasian (CEU), African (YRI), Chinese (CHB) and Japanese (JPT) individuals from the human HapMap genome project (Table 5) and identified 10 distinct SIRPα IgV alleles reflecting combinatorial variation at 18 amino acids (FIG. 8b). A recently reported high-resolution X-ray crystal structure of the SIRPα IgV domain predicts the critical residues that contribute to the CD47 binding surface (Subramanian, S. et al. (2006) Blood Cells Mol Dis 36, 364-72). Strikingly, we observed human allelic variations at predicted CD47 binding residues (FIG. 8b). Moreover, we observed human allelic variations clustered in the same sub-regions of the SIRPα IgV domain that distinguish NOD from NOR alleles (as well as other mouse strains; FIG. 4 and FIG. 8b). Human polymorphism was also seen in the orthologous position to a variation between NOD and NOR (amino acid 72 serine v. asparagine) which may contribute to the distinct patterns of SIRPα N-linked carbohydrate addition we observed in these mouse strains (FIG. 5b and FIG. 8b). Collectively, these data show that, as in laboratory mice, human SIRPα is highly polymorphic in the IgV domain, and suggest that allelic variants will impact posttranslational processing and CD47 ligand interaction.

Our findings identify a new axis of genetically regulated HSC engraftment. Classic graft rejection is an immunologic phenomenon mediated by residual host T cells or NK cells, and rejection rate is related to the degree of HLA disparity between recipient and donor. We show that in addition to T and NK cells, host macrophages play an important role in engraftment mediated through SIRPα-CD47 interactions. Moreover, our findings may be relevant for BM failure syndromes with possible immune-mediated pathogenesis.

It is shown that the effect of mouse macrophages on support of human HSC depends upon SIRPα alleles (is sequence-dependent), and on the number of mouse macrophages in the LTC-IC assays. At high input cell numbers even NOD-derived macrophages reduced support for human HSC in these assays. In clinical settings where the number of human HSC transplanted is limiting and the number of recipient SIRPα bearing macrophages is large, enhanced engraftment could be achieved by treating the recipient with therapeutic doses of CD47-Fc or an equivalent soluble reagent capable of efficient and specific triggering of SIRPα inhibitory signals in recipient macrophages. Once the HSC and their lineage committed progeny engraft and repopulate the recipient, the need for therapeutic engagement of SIRPα might decrease as the established graft produces large numbers of CD47+ cells producing a favorable ratio relative to the remaining recipient-derived macrophages.

A person skilled in the art would understand that one could continue to analyze the degree of human polymorphism in SIRPα by sequencing normal, unrelated individuals, for example, through the available human HapMap samples obtainable through the Sick Kids Centre for Applied Genomics (Toronto, Canada). For example, a 1-2 microgram DNA sample from donor and recipient archival blood samples in the HLA lab would be used for sequencing SIRPα using standard sequencing facilities. The donor/recipient frozen blood sample pairs would be given a unique anonymous ID lacking any diagnosis or any outcome details. Particular attention would be paid to sequence exon 3 of this gene which encodes the IgV domain that is critical to ligand binding and function, and also to sequence two additional exons that encode the intracellular regions of the gene which include important amino acid residue for conveyance of signals emanating from SIRPα. One would create a SIRPα sequence database of these individuals, stripped of any patient identifiers, and perform statistical analysis for association between SIRPα variations and bone marrow engraftment outcomes in the donor-host pairs.

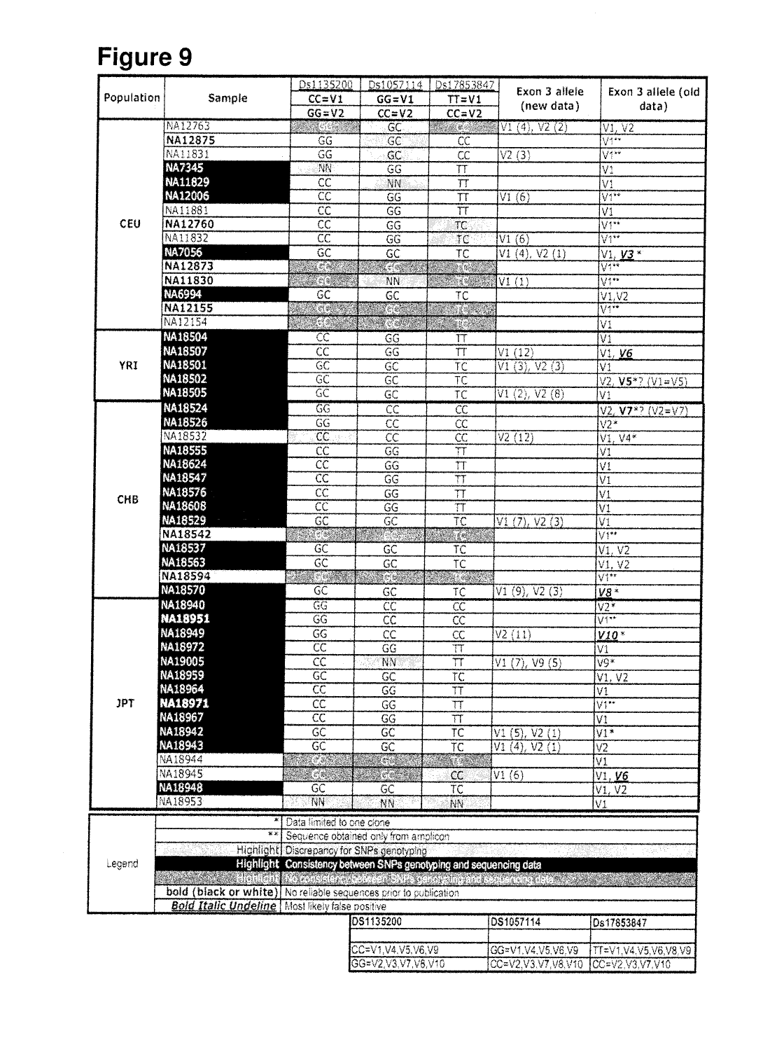

Referring to FIG. 9, three SNP assays (given Ds# notations) were established to detect variable nucleotides underlying several non-synonymous coding polymorphisms we detected among the human HapMap samples. Polymerase chain reaction (PCR) amplification primers and detection probes were designed based on Applied Biosystems TaqMan system. The discrimination between SIRPα variants (V1, V2 . . . etc) is shown at bottom of the Table in FIG. 9. FIG. 9 shows the validation of the discrimination power of each SNP assay by independent sequencing of the SIRPα IgV domain PCR amplified from each of the human HapMap samples shown at the left side of the table. The extent of normal human polymorphism in SIRPα IgV domain sequence is to be determined and use of both SNP assays and sequencing for use in clinical studies is to be optimized. The methods and data employed are widely accessible and known by those of ordinary skill in the art, see for example the International HapMap Project.

### Example 10

Production of hCD47-Fc Protein

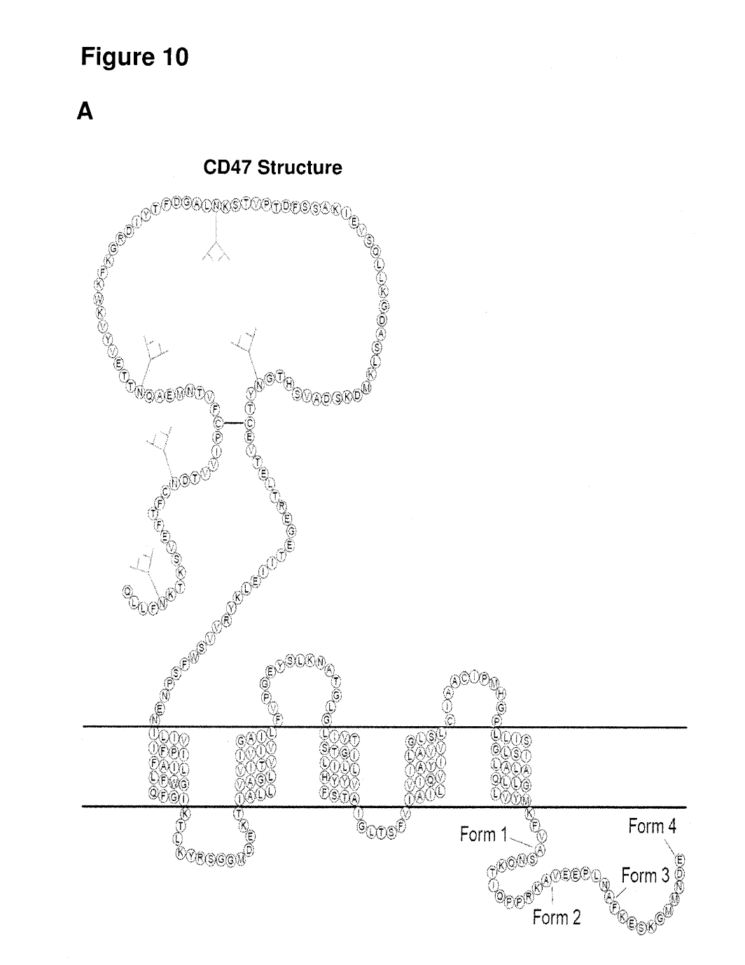

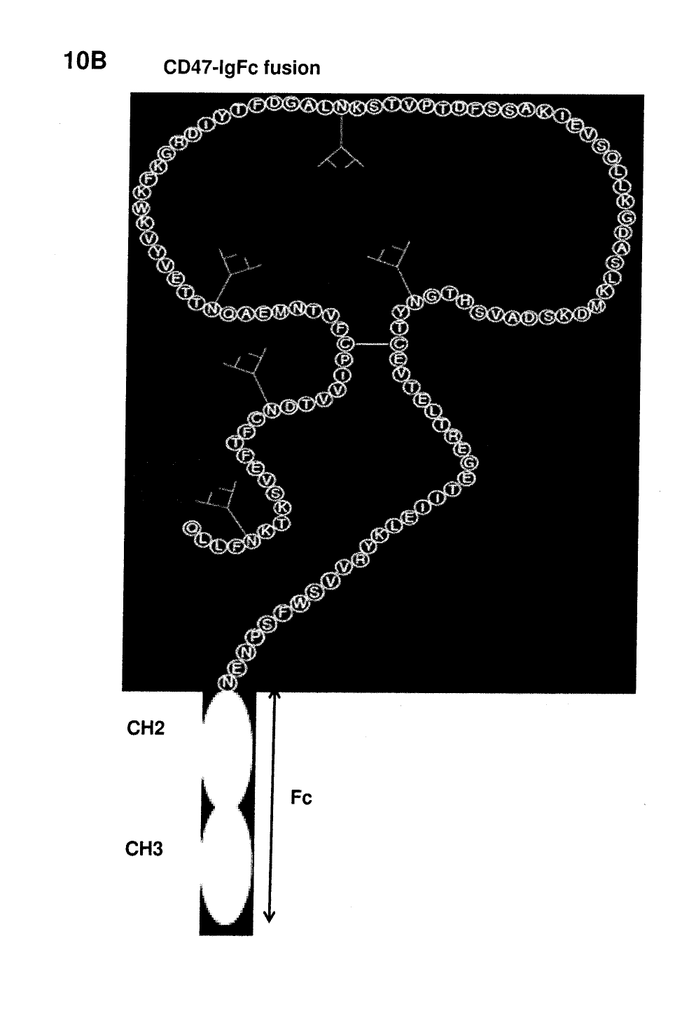

FIG. 10a is a graphic depiction of the CD47 protein including extracellular IgV-like loop, five membrane spanning regions and short cytoplasmic tail. FIG. 10b depicts the CD47-Fc fusion protein representing the CD47 IgV-like domain fused to a human Ig Fc region.

CD47-Fc fusion proteins were prepared as described in the above methods and materials.

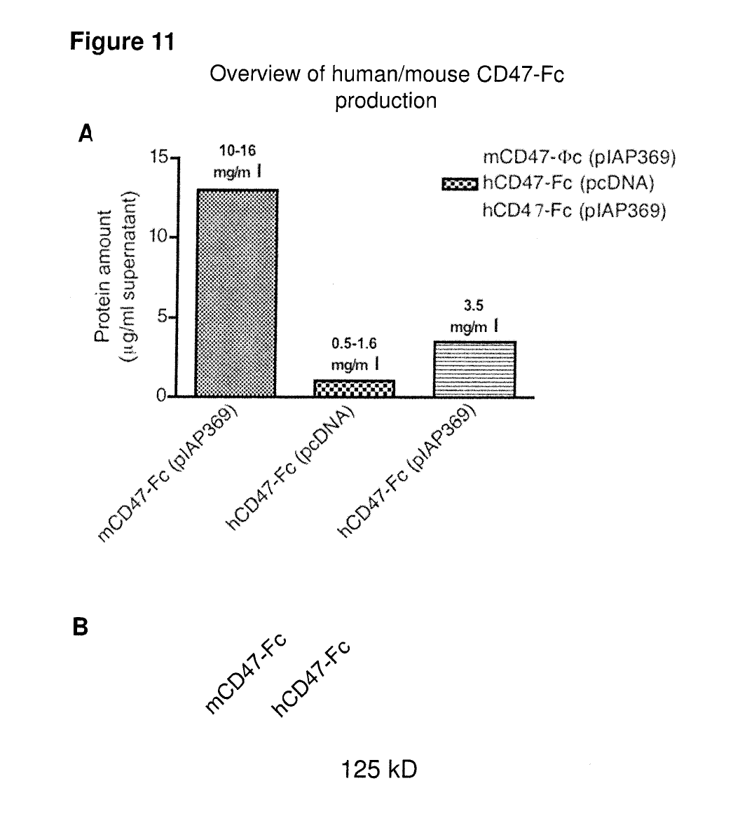

FIG. 11a shows a histogram depicting protein production in the supernatant of cultured cells transfected with mCD47-Fc and two hCD47-Fc constructs prepared with different plasmid backbones (pcDNA and pIAP369). FIG. 11b is an immunoblot of an SDS-PAGE depicting the fusion proteins following purification from culture supernatant. Neither hCD47-Fc construct produced as well as the mCD47-Fc construct prompting further optimization.

### Example 11

Optimization of hCD47-Fc Protein Production

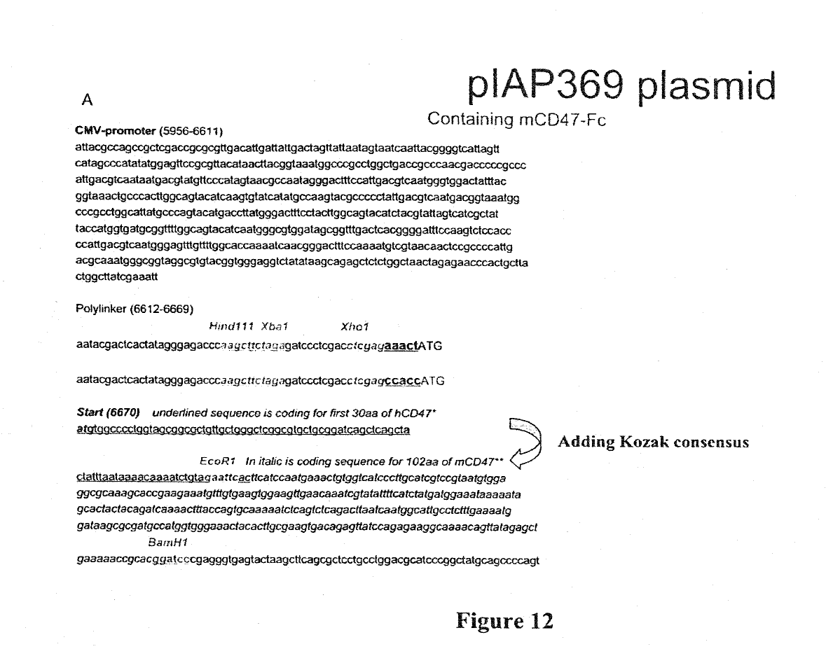

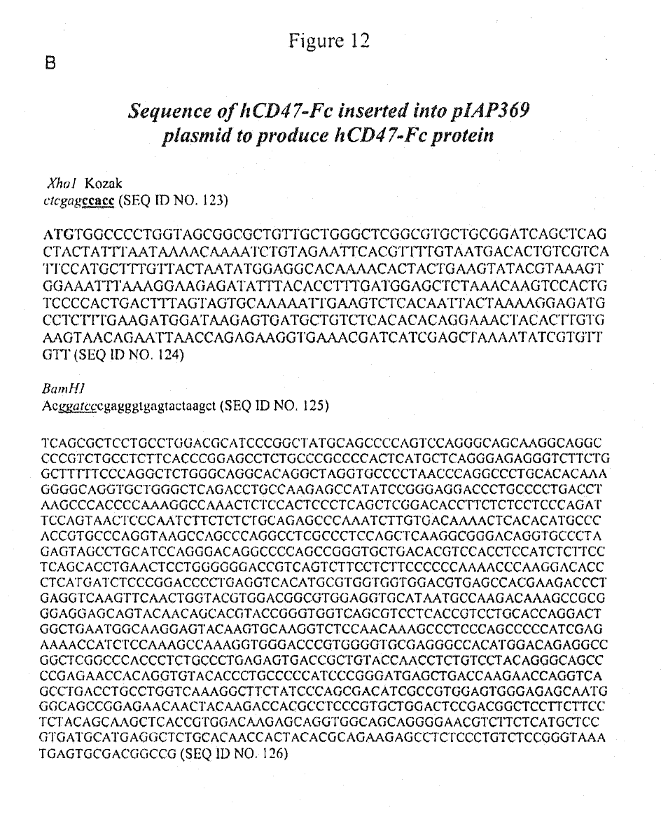

Applicants introduced a consensus initiation site to enhance protein production. Referring to FIG. 12a, the pIAP369 plasmid construct containing mCD47-Fc was used as a scaffold to introduce a eukaryotic translation initiation site (Kozak, M Nucleic Acids Res. 1984, 12(2):857-72) called a Kozak sequence. The resulting plasmid was then digested with restriction enzymes to remove the mCD47 domain which was replaced with the hCD47 IgV-like domain sequence by ligation and re-cloning in bacteria. The resulting plasmid was sequenced to ensure in-frame introduction of the hCD47 sequence and verification of the Kozak sequence. The foregoing utilized typical molecular cloning techniques as would be known to a person of ordinary skill in the art. FIG. 12b shows the sequence hCD47-Fc inserted into the pIAP369 plasmid, showing the Kozak consensus (SEQ ID NO.123), hCD47 fragment (SEQ ID NO.124), linker (SEQ ID NO.125) and Fc (SEQ ID NO.126).

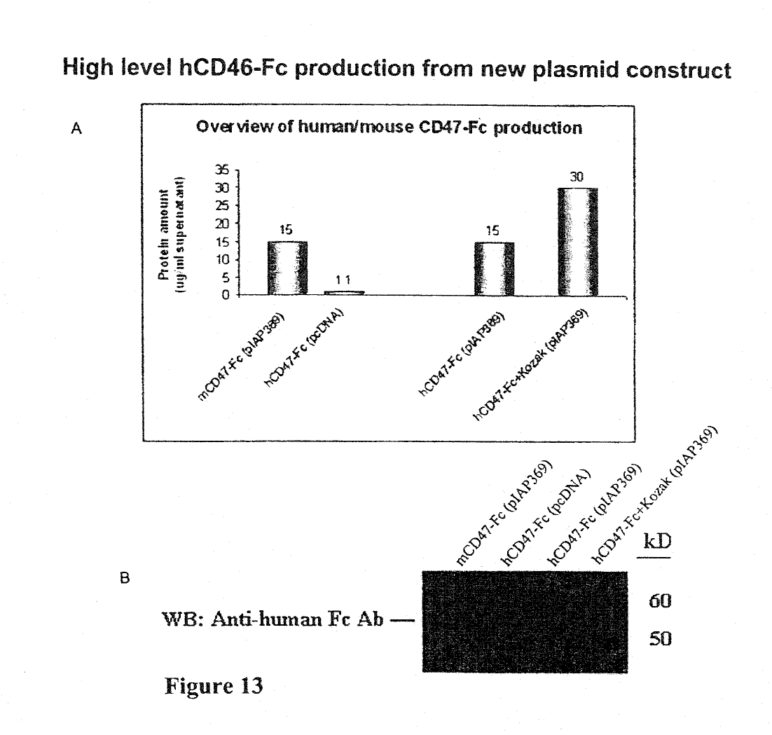

FIG. 13a shows the production of mouse and human CD47-Fc assayed in the supernatant of cells transfected with one of four different constructs prepared as above. The data show that introduction of the eukaryotic initiation (Kozak) sequence enhanced production of hCD47-Fc (compare two right hand columns) to 30 mg/L of supernatant. FIG. 13b shows immunoblot displaying an anti-human Fc antibody reacting with the protein G-purified CD47-Fc proteins. Note that differential post-translational additional of carbohydrate moieties produces differences in SDS-PAGE mobilities for mouse and human CD47-Fc.

Applicant has succeeded in producing significant amounts of highly purified CD47-Fc fusion proteins suitable for in vivo testing in the NOD.SCID xenotransplant model.

### Example 12

### Mouse CD47-Fc Binding to NOD and NOR SIRPα

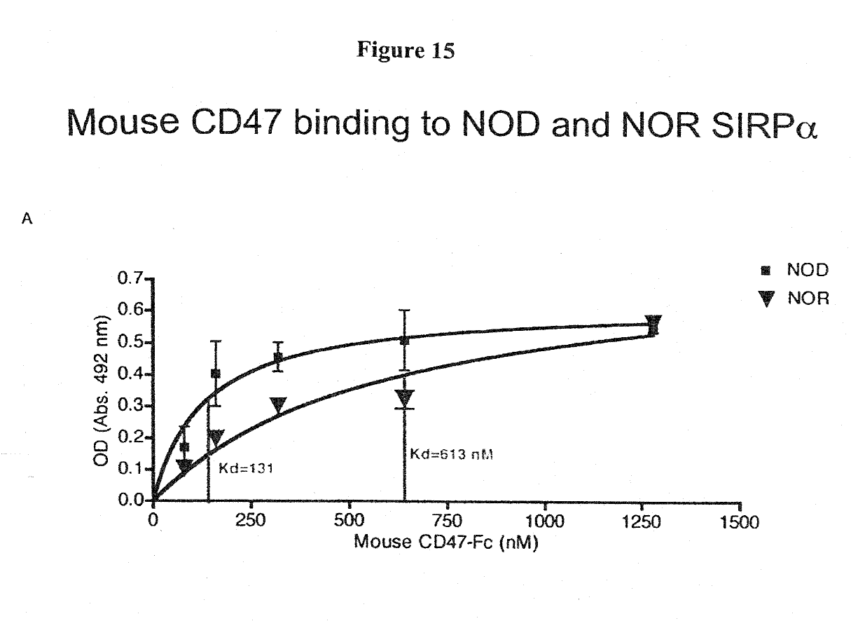

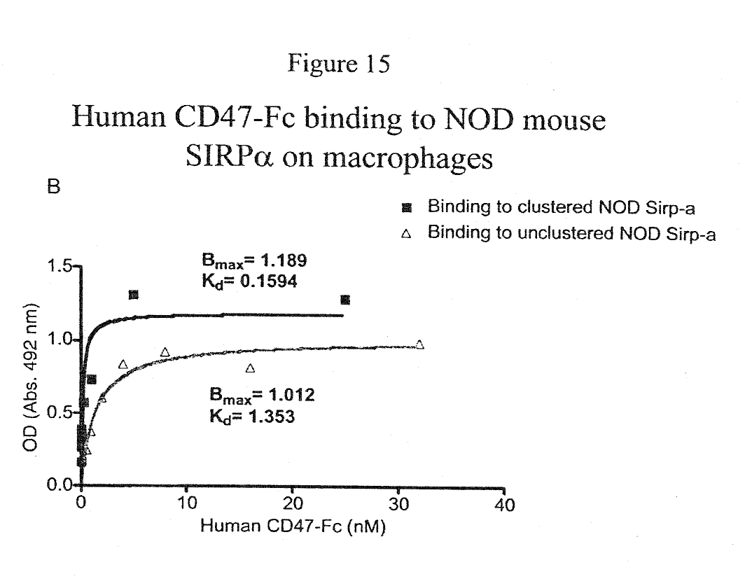

Assay of mCD47-Fc binding to SIRPα on the surface of freshly prepared NOD and NOR macrophages was determined using the assay described in the materials and methods section in relation to FIG. 14. FIG. 15 shows the results that the Kd of NOD SIRPα-mCD47 binding is with a greater than 4 fold (about 4.7 fold) less than the Kd of NOR SIRPα-mCD47 interaction representing a greater than 4 fold higher affinity of interaction by the NOD compared to the NOR variant of SIRPα. These data suggest that the SIRPα variations we have observed in human SIRPα IgV domain sequence is predicted to confer significant differences in binding to human CD47 which may affect the efficacy of HSC transplantation.

### Example 13

### Human CD47-Fc Binding to NOD and NOR SIRPα

Assay of hCD47-Fc binding to SIRPα on the surface of freshly prepared NOD and NOR macrophages was determined using the assay described in the materials and methods section in relation to FIG. 14. Two conditions were tested: first, binding to SIRPα on unmanipulated fresh macrophages (“unclustered”; triangles) and second, to macrophages that had been pre-treated with a monoclonal antibody to SIRPα ectodomain (“clustered”; squares). The latter condition was examined to mimic the effect of engaging CD47 displayed on the surface of human HSC and their progeny cells. In that setting, the CD47 binding would create a higher valency, “clustering” the bound SIRPα proteins on the mouse macrophage. In FIG. 15b, the results show that the Kd of NOD SIRPα-hCD47 binding is 1.6 nM, representing a high affinity interaction. Strikingly, under the clustering conditions, the affinity rise by more than 10-fold providing a Kd of 0.16 nM. These data suggest that the human CD47 binding to NOD-derived SIRPα IgV domain is very high and predictive of a strong therapeutic effect of the CD47-Fc fusion proteins in vivo.

### Example 14

### Evaluation of the Role of SIRPα-Mediated Signal Transduction on Engraftment of HSC

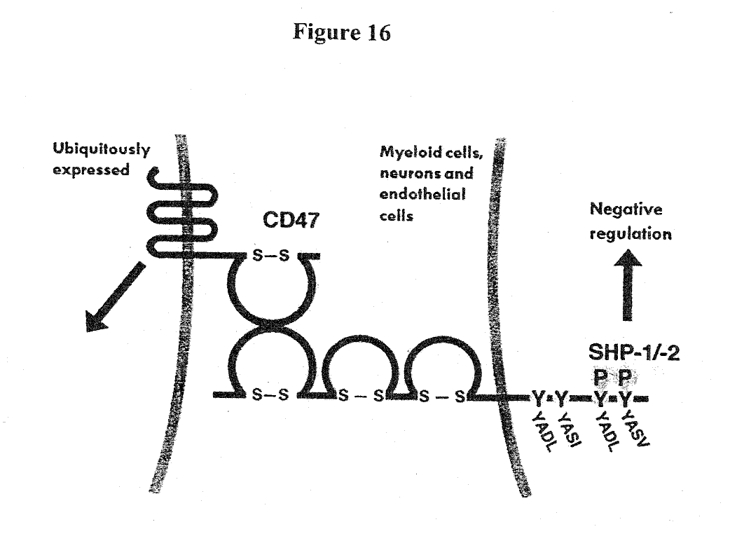

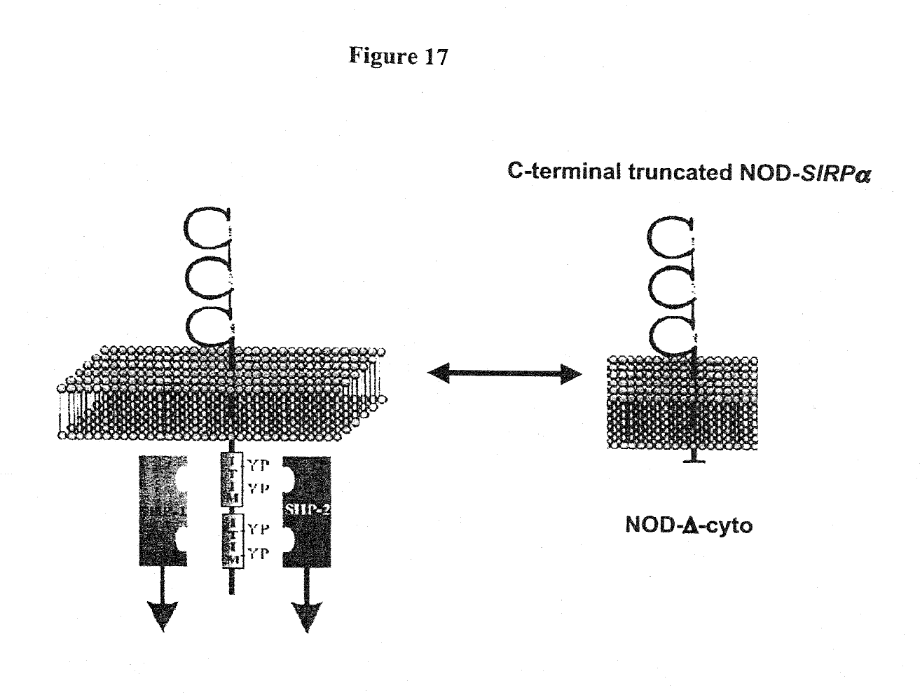

Other studies suggest that SIRPα-CD47 binding elicits bi-directional signals through each of the proteins (represented by the schematic shown in FIG. 16). Applicants evaluated whether signals through SIRPα, mediated by CD47 binding, are required for HSC engraftment. FIG. 17 shows preparation of a truncated version of the human HSC “permissive” NOD version of SIRPα (“NOD-Δ-cyto”) (left side) incapable of transmitting signals by intentional deletion of the two immuno tyrosine inhibitory motifs (ITIM) required to link to critical downstream phosphatases SHP-1 and SHP-2 (right side).

A long-term culture initiating cell (LTC-IC) assay (performed using method described in Takenaka et al Nature Immunol 2007 December, 8(12): 1313-23) to determine requirement for SIRPα-CD47 mediated signaling in human HSC survival. This experiment compares the capacity of full length cytoplasmic truncated NOD SIRPα to support human HSC survival in LTC-IC assays. Five conditions were tested using graded doses of mouse macrophages of either NOD or NOR origin transduced with different lentiviruses:
1) NOR macrophages infected with an “empty” lentivirus (NOR-CEP “diamonds”)
2) NOD macrophages not infected with virus (NOD uninf; filled triangles)
3) NOD macrophages infected with “empty” lentivirus (NOD-CEP; circles)
4) NOR macrophages infected with lentivirus containing full length NOD SIRPα (NOR-SIRP; squares)
5) NOR macrophages infected with lentivirus containing truncated NOD SIRPα (NOD-α-cyto; open triangles)

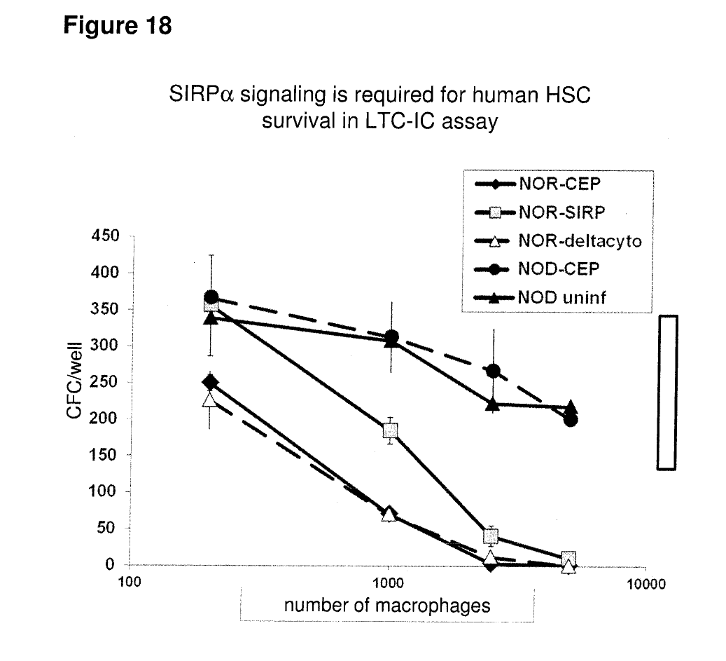

Referring to FIG. 18, infection of NOR macrophages with lentivirus containing a full length NOD SIRPα construct significantly enhances human HSC survival compared to NOR macrophages infected with the empty virus (squares v. diamonds). In contrast, infection of NOR macrophages with a lentivirus containing the cytoplasmic truncated version of NOD SIRPα is not distinguishable from infection of NOR macrophages with empty virus (diamonds v. open triangles). These data show that support of human HSC depends upon CD47-dependent signaling through SIRPα. Therefore, therapeutics development to enhance human HSC transplantation, survival and differentiation in patients should focus on manipulation of signals through SIRPα. Applicants reasoned that the NOD.SCID xenotransplantation data reported in Takenaka et al (supra) emulate clinical settings where the patient receiving a small population of HSC has abundant macrophages expressing SIRPα. In this “non-myeloablative” setting, binding affinity between CD47 on the donor HSC and SIRPα expressed on the patient's macrophages could control the efficiency of triggering SIRPα-mediated signals and could determine whether the HSC persisted or eliminated.

### Example 15

Evaluation In Vivo of hCD47-Fc Therapy in Human HSC Engraftment

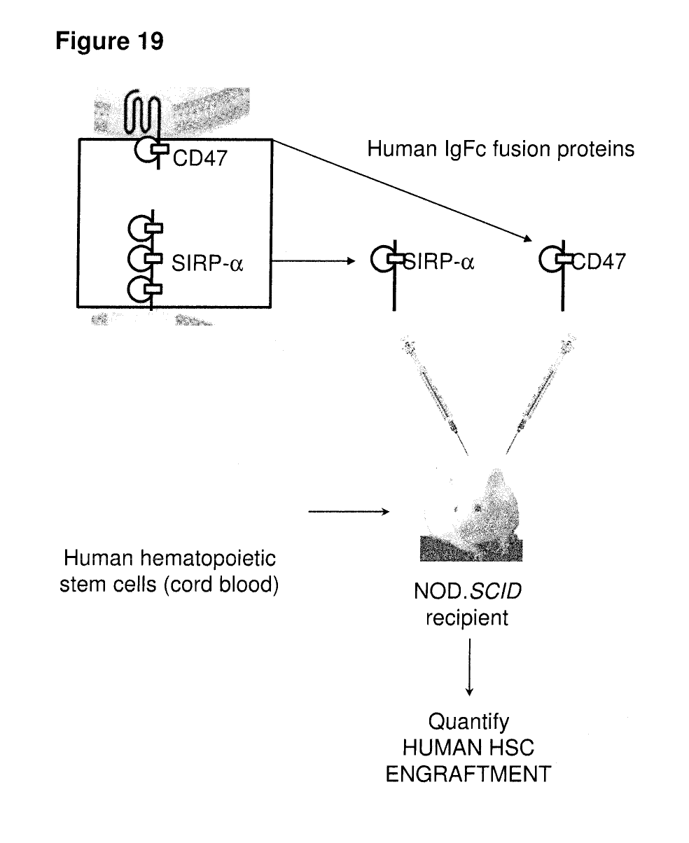

FIG. 19 is a general outline of a study designed to evaluate in vivo the effect of CD47 fusion protein on HSC engraftment. The hCD47-Fc fusion protein was injected into NOD.SCID hosts together with introduction of graded doses of human newborn cord blood hematopoietic stem cells. Human HSC were prepared by removal of mature lineage-committed cells using a cocktail of monoclonal antibodies and immunomagnetic beads as previously described (J. L. McKenzie et al., Nat Immunol. 2006, 11:1225-33). HSC engraftment is followed by flow cytometric examination with human-specific antibodies to cell surface markers (CD34, CD45) in blood, spleen and bone marrow of the recipient mice.

### Human CD47-Fc Fusion Protein Confers Enhanced Engraftment of Human HSC

A study performed was to determine whether treatment with human CD47-Fc fusion protein would influence engraftment of human cord blood stem cells upon transplantation into NOD.SCID mice. Human newborn cord blood cells were depleted with a cocktail of antibodies against blood cell lineage markers using our previously published methods (Vormoor J, Lapidot T, Pflumio F, Risdon G, Patterson B, Broxmeyer H E, Dick J E. Blood. 1994, 83(9):2489-97) resulting in an enriched populations of HSC. Cohorts of age matched female NOD.SCID mice were pre-conditioned with low dose irradiation as we previously described (Vormoor J, Lapidot T, Pflumio F, Risdon G, Patterson B, Broxmeyer H E, Dick J E. Blood. 1994, 83(9):2489-97). Twenty to forty thousand lineage depleted CB cells were injected into NOD.SCID mice that were injected intraperitoneally with either purified human CD47-Fc protein or an irrelevant human Fc isotype control preparation. The treatment was administered three hours before injection of the CB cells, and then 3 times/week for 3 weeks (7.5-30 micrograms/dose). At the conclusion of the experiment, all mice were sacrificed and bone marrow cells prepared from the tibia and femur. The bone marrow cells were stained with a series of monoclonal antibodies and analyzed by flow cytometry. Cells staining with anti-human CD45 were quantified to determine the extent of human HSC engraftment.

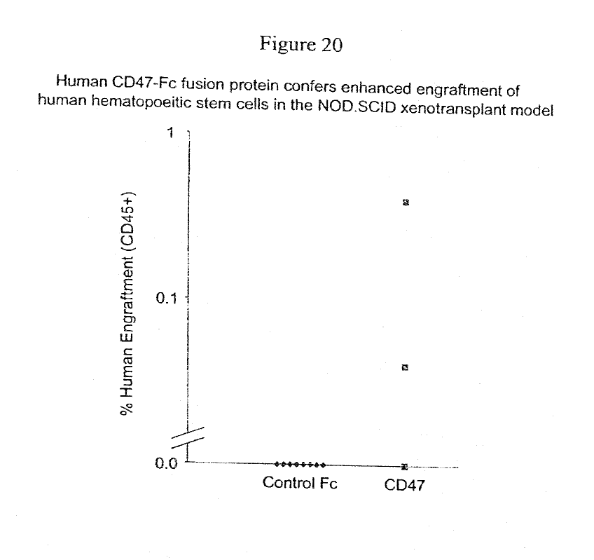

Referring to FIG. 20, Human CD45+ cells were not detectable in eight NOD.SCID mice that received the control human Fc protein. In contrast, two of three mice treated with hCD47-Fc protein displayed clear evidence of human HSC engraftment. These data are consistent with the idea that treatment with protein or small molecule agonists of SIRPα can promote engraftment of human HSC in settings of clinical transplantation.

Although preferred embodiments of the invention have been described herein, it will be understood by those skilled in the art that variations may be made thereto without departing from the spirit of the invention or the scope of the appended claims. All documents disclosed herein are incorporated by reference.

We claim:

1. A method for increasing hematopoietic stem cell engraftment in a human in need thereof, comprising administering a therapeutically effective amount of a protein capable of binding to the extracellular domain of human Sirpα.

2. The method of claim 1, wherein the protein comprises soluble human CD47, or a fragment thereof.

3. The method of claim 2, wherein the protein is the extracellular domain of human CD47,

4. The method of claim 3, wherein the protein is the extracellular domain of human CD47 fused to a second protein.

5. The method of claim 4, wherein the protein is the extracellular domain of human CD47 fused to the Fc portion of IgG.

6. The method of claim 1, wherein the protein is an antibody to human Sirpα.

7. The method of claim 1, wherein the method further results in suppression of macrophages.

8. A polypeptide comprising the extracellular domain of CD47 fused to the Fc portion of IgG.

9. The polypeptide of 8, wherein the said polypeptide is encoded consecutively by SEQ ID NOs. 124, 125 and 126.

10. A pharmaceutical composition comprising a pharmaceutically acceptable carrier and a polypeptide selected from a polypeptide comprising the extracellular domain of CD47 fused to the Fc portion of IgG and a polypeptide comprising the extracellular domain of CD47 fused to the Fc portion of IgG encoded consecutively by SEQ ID NOs: 124, 125 and 126.


SEQUENCE LISTING (126 sequences)

SEQ ID NO: 1 - protein, 507 aa, Mouse
MEPAGPAPGRLGPLLLCLLLSASCFCTGATRTEVKVIQPEKSVSVAAGDSTVLNCTLTSLLPVGPIRWYRGVGQSRQLIYSFTTEHFPRVTNVSDATKRSNLDFSIRISNVTPEDAGTYYCVKFQRGSPDTEIQSGGGTEVYVLAKPSPPEVSGPADRGIPDQKVNFTCKSHGFSPRNITLKWFKDGQELHPLETTVNPSGKNVSYNISSTVRVVLNSMDVHSKVICEVAHITLDRSPLRGIANLSNFIRVSPTVKVTQQSPTSMSQVNLTCRAERFYPEDLQLIWLENGNVSRNDTPKNLTKNTDGTYNYTSLFLVNSSAHREDVVFTCQVKHDQQPAITRNHTVLGLAHSSDQGSMQTFPGNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 2 - protein, 509 aa, Mouse
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 3 - protein, 513 aa, Mouse
MEPAGPAPGRLGPLLLCLLLSASCFCTGATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLAKPSPPEVSGPADRGIPDQKVNFTCKSHGFSPRNITLKWFKDGQELHPLETTVNPSGKNVSYNISSTVRVVLNSMDVNSKVICEVAHITLDRSPLRGIANLSNFIRVSPTVKVTQQSPTSMNQVNLTCRAERFYPEDLQLIWLENGNVSRNDTPKNLTKNTDGTYNYTSLFLVNSSAHREDVVFTCQVKHDQQPAITRNHTVLGFAHSSDQGSMQTFPDNNATHNWNVFIGVGVACALLVVLLMAALYLLRIKQKKAKGSTSSTRLHEPEKNAREITQVQSLIQDTNDINDITYADLNLPKEKKPAPRAPEPNNHTEYASIETGKVPRPEDTLTYADLDMVHLSRAQPAPKPEPSFSEYASVQVQRK

SEQ ID NO: 4 - protein, 120 aa, Human
GVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 5 - protein, 119 aa, Human
GVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRA

SEQ ID NO: 6 - protein, 119 aa, Human
GVAGEEELQVIQPDKSVSVAAGESAILLCTVTSLIPVGPIQWFRGAGPARELIYNQKEGHFPRVTTVSESTKRENMDFSISISNITPADAGTYYCVKFRKGSPDTEFKSGAGTELSVRA

SEQ ID NO: 7 - protein, 120 aa, Human
GVAGEEGLQVIQPDKSVSVAAGESAILHCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 8 - protein, 120 aa, Human
GVAGEEELQVIQPDKFVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 9 - protein, 120 aa, Human
GVAGEEELQVIQPDKSVLVAAGETATLRCTATSLIPVGPIQWFRGAGPGRELIYNQKEGHFPRVTTVSDLTKRNNMDFPIRIGNITPADAGTYYCVKFRKGSPDDVEFKSGAGTELSVRA

SEQ ID NO: 10 - protein, 120 aa, Human
GVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDDVEFKSGAGTELSVRA

SEQ ID NO: 11 - protein, 119 aa, Human
GVAGEEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRA

SEQ ID NO: 12 - protein, 120 aa, Human
GVAGEEELQVIQPDKSVLVAAGETATLRCTATSLIPVGPIQWFRGAGPGRELIYNQKEGHFPRVTTVSDLTKRNNMDFSIRISNITPADAGTYYCVKFRKGSPDDVEFKSGAGTELSVRA

SEQ ID NO: 13 - protein, 119 aa, Human
RVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRA

SEQ ID NO: 14 - protein, 119 aa, Mouse
ATGKELKVTQPEKSVSVAAGDSTVLNCTLTSLLPVGPIRWYRGVGPSRLLIYSFAGEYVPRIRNVSDTTKRNNMDFSIRISNVTPADAGIYYCVKFQKGSSEPDTEIQSGGGTEVYVLA

SEQ ID NO: 15 - protein, 119 aa, Mouse; identical to SEQ ID NO: 14

SEQ ID NO: 16 - protein, 117 aa, Mouse
ATRTEVKVIQPEKSVSVAAGDSTVLNCTLTSLLPVGPIRWYRGVGQSRQLIYSFTTEHFPRVTNVSDATKRSNLDFSIRISNVTPEDAGTYYCVKFQRGSPDTEIQSGGGTEVYVLA

SEQ ID NO: 17 - protein, 119 aa, Mouse
ATGTEVKVTQPEKSVSVAAGDSTILNCTVTSLLPVGPIRWYRGVGQSRLLIYSFTGEHFPRVRNVSDTTKRNNMDFSIRISNVTPEDAGTYYCVKFQRGSSEPDTEIQSGGGTEVYVLA

SEQ ID NO: 18 - protein, 119 aa, Mouse
VTGKELKVTQPEKSVSVAAGDSTVLNCTLTSLLPVGPIKWYRGVGQSRLLIYSFTGEHFPRVTNVSDATKRNNMDFSIRISNVTPEDAGTYYCVKFQKGPSEPDTEIQSGGGTEVYVLA

SEQ ID NO: 19 - DNA, 23 bp, Mouse
gaacataggtgcacactcacata

SEQ ID NO: 20 - DNA, 19 bp, Mouse
tgatgctggaggttgtcct

SEQ ID NO: 21 - DNA, 29 bp, Mouse
gatgaaagagataagacagatgttagata

SEQ ID NO: 22 - DNA, 26 bp, Mouse
tggttattaaagagctgttttacatg

SEQ ID NO: 23 - DNA, 21 bp, Mouse
aagttggaactctgcaggaca

SEQ ID NO: 24 - DNA, 20 bp, Mouse
gtgtcttcaatgcagcacgt

SEQ ID NO: 25 - DNA, 23 bp, Mouse
tattgtttatggaaattggaccc

SEQ ID NO: 26 - DNA, 22 bp, Mouse
catctctgaaggaaaaagtgca

SEQ ID NO: 27 - DNA, 25 bp, Mouse
gggagacaacaaataatcatattgc

SEQ ID NO: 28 - DNA, 20 bp, Mouse
agaggtgggtcctggaaact

SEQ ID NO: 29 - DNA, 20 bp, Mouse
ctgaaaatcgtcccttgacc

SEQ ID NO: 30 - DNA, 20 bp, Mouse
caggagcatgaaatggggat

SEQ ID NO: 31 - DNA, 20 bp, Mouse
tccacacaaggtgtcctctg

SEQ ID NO: 32 - DNA, 20 bp, Mouse
gctcaggtgacctccaaaac

SEQ ID NO: 33 - DNA, 24 bp, Mouse
atcaccactgtgggcattctgaac

SEQ ID NO: 34 - DNA, 26 bp, Mouse
cagtagaggccaagggcatcagaatc

SEQ ID NO: 35 - DNA, 20 bp, Mouse
tgtctccccaaagtagcagg

SEQ ID NO: 36 - DNA, 20 bp, Mouse
gtgatgcaggagtttctgca

SEQ ID NO: 37 - DNA, 20 bp, Mouse
ggcagatttatcgctgtgag

SEQ ID NO: 38 - DNA, 15 bp, Mouse
gtgcgccaccaatgc

SEQ ID NO: 39 - DNA, 21 bp, Mouse
cttcagtgctgtatccactgc

SEQ ID NO: 40 - DNA, 21 bp, Mouse
ctcccactcccattaattctc

SEQ ID NO: 41 - DNA, 19 bp, Mouse
aggcaattacaaggcctgg

SEQ ID NO: 42 - DNA, 20 bp, Mouse
cacccatctccctcagtcat

SEQ ID NO: 43 - DNA, 22 bp, Mouse
gtagagacttggtgggttttgg

SEQ ID NO: 44 - DNA, 23 bp, Mouse
atctctgctgacaaagaacagtg

SEQ ID NO: 45 - DNA, 20 bp, Mouse
catgtgccatggtacaaagc

SEQ ID NO: 46 - DNA, 21 bp, Mouse
tggcttgtttcagatagcaca

SEQ ID NO: 47 - DNA, 20 bp, Mouse
tcaccagcctgaaaacactg

SEQ ID NO: 48 - DNA, 23 bp, Mouse
tctgggtacaatccttagtcctg

SEQ ID NO: 49 - DNA, 21 bp, Mouse
gccagatacaattttccctca

SEQ ID NO: 50 - DNA, 18 bp, Mouse
gtgggacgcaatattggc

SEQ ID NO: 51 - DNA, 20 bp, Mouse
tcccacatttctggcataca

SEQ ID NO: 52 - DNA, 21 bp, Mouse
cacatgtgcatttaagcatgc

SEQ ID NO: 53 - DNA, 18 bp, Mouse
gtgaggggtcaatgccac

SEQ ID NO: 54 - DNA, 21 bp, Mouse
ggctcagttgtaagcacaagg

SEQ ID NO: 55 - DNA, 18 bp, Mouse
cctaaccctccccacacc

SEQ ID NO: 56 - DNA, 23 bp, Mouse
aaagatctggattcaaatcctcc

SEQ ID NO: 57 - DNA, 22 bp, Mouse
acgagttgtcctctgatcaaca

SEQ ID NO: 58 - DNA, 20 bp, Mouse
agccagagcaaacaccaact

SEQ ID NO: 59 - DNA, 21 bp, Mouse
tatggatccactctccagaaa

SEQ ID NO: 60 - DNA, 20 bp, Mouse
caaagtctcctccaaggctg

SEQ ID NO: 61 - DNA, 19 bp, Mouse
ctgctgcagcgattctctc

SEQ ID NO: 62 - DNA, 22 bp, Mouse
tcaggcacctaagtacatgtgc

SEQ ID NO: 63 - DNA, 26 bp, Mouse
aaaaatcgttctttgacttctacatg

SEQ ID NO: 64 - DNA, 25 bp, Mouse
tttaaaagggtttctttatcctgtg

SEQ ID NO: 65 - DNA, 25 bp, Mouse
ttttcttagaacataccttgcttgg

SEQ ID NO: 66 - DNA, 22 bp, Mouse
ggggaaggtgaaaatttaaagg

SEQ ID NO: 67 - DNA, 20 bp, Mouse
tctccacgtgtgtgccttag

SEQ ID NO: 68 - DNA, 21 bp, Mouse
tgaaagcactctgcagactca

SEQ ID NO: 69 - DNA, 20 bp, Mouse
cactacacggggctgagatt

SEQ ID NO: 70 - DNA, 21 bp, Mouse
aagatccaacaccctttttgg

SEQ ID NO: 71 - DNA, 20 bp, Mouse
ggcagctttgtgctttaaaa

SEQ ID NO: 72 - DNA, 19 bp, Mouse
atggatgtttacctggcgt

SEQ ID NO: 73 - DNA, 26 bp, Mouse
actggaaaatatgttttaaacctctg

SEQ ID NO: 74 - DNA, 21 bp, Mouse
aaatgggattctgcaaaaacc

SEQ ID NO: 75 - DNA, 20 bp, Mouse
gtgcactttccatgcctgta

SEQ ID NO: 76 - DNA, 27 bp, Mouse
gagtagaaagagacagagaaagacaca

SEQ ID NO: 77 - DNA, 20 bp, Mouse
tctccagccccttcattatg

SEQ ID NO: 78 - DNA, 19 bp, Mouse
tgccaaacacccatgagac

SEQ ID NO: 79 - DNA, 20 bp, Mouse
tccttggtgagtccacatct

SEQ ID NO: 80 - DNA, 19 bp, Mouse
ggctaggctctggaagaca

SEQ ID NO: 81 - DNA, 23 bp, Mouse
actagccatatggtttctgatgg

SEQ ID NO: 82 - DNA, 20 bp, Mouse
gtagcagggctgtgagcttt

SEQ ID NO: 83 - DNA, 23 bp, Mouse
tattaatgttgaagccagaagcc

SEQ ID NO: 84 - DNA, 23 bp, Mouse
ctttaatcatctctgtggcaagg

SEQ ID NO: 85 - DNA, 23 bp, Mouse
tgaagcaagaattcaagaagagg

SEQ ID NO: 86 - DNA, 25 bp, Mouse
aggacatacctagtaaggctttgtc

SEQ ID NO: 87 - DNA, 23 bp, Mouse
gatcttcctaccttcttacccac

SEQ ID NO: 88 - DNA, 19 bp, Mouse
catgattttatttgggggg

SEQ ID NO: 89 - DNA, 22 bp, Mouse
gtcagacattggcttaggatcc

SEQ ID NO: 90 - DNA, 20 bp, Mouse
ggtttgtgcgctctctctct

SEQ ID NO: 91 - DNA, 20 bp, Mouse
tttgcctgtaacaagccaga

SEQ ID NO: 92 - DNA, 24 bp, Mouse
ttgaggctatcagtttaaaatccc

SEQ ID NO: 93 - DNA, 24 bp, Mouse
gaataacccatgtttatatcggtg

SEQ ID NO: 94 - DNA, 22 bp, Mouse
ctctggacttgtgttctatgcc

SEQ ID NO: 95 - DNA, 21 bp, Mouse
cctcctaatagtgccattccc

SEQ ID NO: 96 - DNA, 20 bp, Mouse
ccagcaatcacgagtcttcc

SEQ ID NO: 97 - DNA, 24 bp, Mouse
gaccaaagtcatatctataacacc

SEQ ID NO: 98 - DNA, 27 bp, Mouse
ttaattgcaagtaacacaatgagtagg

SEQ ID NO: 99 - DNA, 18 bp, Mouse
actgttgctggggaatgg

SEQ ID NO: 100 - DNA, 19 bp, Mouse
ccaagttcaaagctgctgg

SEQ ID NO: 101 - DNA, 26 bp, Mouse
agtccaccttaagaggaccaagtagc

SEQ ID NO: 102 - DNA, 21 bp, Mouse
tgtacagaaacaggacgcgga

SEQ ID NO: 103 - DNA, 20 bp, Mouse
tctccctccttgctctgcag

SEQ ID NO: 104 - DNA, 26 bp, Mouse
tacctcgcagatgaccttagaattaa

SEQ ID NO: 105 - DNA, 28 bp, Mouse
cctagtggaaagaatgtctcctacaaca

SEQ ID NO: 106 - DNA, 26 bp, Mouse
catattctgtgtggttgttaggctca

SEQ ID NO: 107 - DNA, 24 bp, Mouse
ccaggtacagtctttgatccagga

SEQ ID NO: 108 - DNA, 22 bp, Mouse
ccagggagtctctgctggtcta

SEQ ID NO: 109 - DNA, 28 bp, Mouse
catctgtggaagaactacaccagaagtc

SEQ ID NO: 110 - DNA, 22 bp, Mouse
tacctcagagagctggccgagt

SEQ ID NO: 111 - DNA, 22 bp, Mouse
ccaatgctgacctaatgttggc

SEQ ID NO: 112 - DNA, 28 bp, Mouse
catctatacacccgtgtgtgtatacaca

SEQ ID NO: 113 - DNA, 20 bp, Mouse
aacttgtctttgtccgggcc

SEQ ID NO: 114 - DNA, 20 bp, Mouse
ggcacagttcatcctcaccc

SEQ ID NO: 115 - DNA, 25 bp, Human
tagaatacaggctcatgttgcaggt

SEQ ID NO: 116 - DNA, 23 bp, Human
gccttcagcaaatagcatgacgt

SEQ ID NO: 117 - DNA, 20 bp, Mouse
gccactaccatccgattctt

SEQ ID NO: 118 - DNA, 20 bp, Mouse
cctgtcttgaaaacccaaaa

SEQ ID NO: 119 - DNA, 20 bp, Mouse
aatgacttgtttgccaagga

SEQ ID NO: 120 - DNA, 20 bp, Mouse
ttttatgccctaggagcctc

SEQ ID NO: 121 - DNA, 21 bp, Mouse
actctctgagtagcctgcccc

SEQ ID NO: 122 - DNA, 20 bp, Mouse
cctcacccttacttggcacc

SEQ ID NO: 123 - DNA, 11 bp, Human
ctcgagccacc

SEQ ID NO: 124 - DNA, 402 bp, Human
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtt

SEQ ID NO: 125 - DNA, 27 bp, Human
acggatcccgagggtgagtactaagct

SEQ ID NO: 126 - DNA, 1267 bp, Human
tcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccg